(19) United States
(12) Patent Application Publication
(10) Pub. No.: US 2008/0318838 A1
(43) Pub. Date: Dec. 25, 2008

(54) PEPTIDES

(76) Inventors: Wilfried Bauer, Lampenberg (CH); Robin Breckenridge, Muttenz (CH); Francois Cardinaux, Seewen (CH); Frank Gombert, Huttingen (DE); Hermann Gram, Weil-Haltingen (DE); Paul Ramage, Reinach (CH); Helmut Schneider, Frieburg (DE); Rudolf Waelchli, Basel (CH); Rainer Albert, Basel (CH); Ian Lewis, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/169,134

(22) Filed: Jul. 8, 2008

Related U.S. Application Data
(63) Continuation of application No. 10/848,279, filed on May 18, 2004, now abandoned.
Application No. 10/848,279 is a continuation of application No. 08/466,487, filed on Jun. 6, 1995, now abandoned.
Application No. 08/466,487 is a continuation of application No. 08/345,151, filed on Nov. 28, 1994, now abandoned.
Application No. 08/345,151 is a continuation of application No. 08/158,744, filed on Nov. 30, 1993, now abandoned.
Application No. 08/158,744 is a continuation of application No. 08/091,503, filed on Jul. 14, 1993, now abandoned.

(30) Foreign Application Priority Data
Jul. 15, 1992 (GB) 9215009.3
Dec. 18, 1992 (GB) 9226415.9
Dec. 23, 1992 (GB) 9226859.8
Dec. 23, 1992 (GB) 9226861.4
Jan. 28, 1993 (GB) 9301691.3
Jan. 28, 1993 (GB) 9301692.1
Apr. 14, 1993 (GB) 9307673.5
Apr. 19, 1993 (GB) 9308033.1

(51) Int. Cl. A61K 38/00; C07K 7/00; C12P 21/04; C12N 15/11; A61P 19/00; C12N 15/00; C12N 1/20

(52) U.S. Cl. 514/2; 530/300; 435/69.4; 536/23.4; 435/320.1; 435/252.3

(57) ABSTRACT

PTH compounds having PTH-like activity and comprising at least one modification, said modification being either
1. at least one radical selected from a L- or D-α-amino acid, C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to the terminal amino group of the PTH compound, and/or at least one radical selected from C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to one or more side chain amino groups of the PTH compound,
or
2. at least one α-amino acid unit in the positions 1 to 38 of a naturally occurring PTH sequence being replaced by a natural or unnatural amino acid unit optionally in protected form, whereby the α-amino acid units present in positions 1 and 2 at the amino terminus of the PTH sequence may be replaced by a pseudo-peptide,
or a combination of such modifications,
   in free form or in salt form, have pharmacological activity, e.g. for preventing or treating all bone conditions which are associated with increased calcium depletion or resorption or in which calcium fixation in the bone is desirable.

The present invention relates to variants of parathyroid hormone (PTH), a process for their production, pharmaceutical preparations comprising them and their use as a pharmaceutical.

The term “PTH” as used herein refers to any genetically encoded form of parathyroid hormone, including the mature form containing 84 amino acids of a given vertebrate PTH species, e.g. human, porcine, rat, bovine, chicken, and fragments thereof as well as analogues and derivatives thereof having PTH-like activity. The position of each amino acid involved in the PTH sequence is numbered according to the internationally accepted procedure. For consistency and as is conventional, in the following description, the same numbering system will be applied to the amino acids of the PTH sequence independently of the substitution pattern in the molecule.

More particularly, the present invention provides a PTH compound having PTH-like activity and comprising at least one modification, said modification being either
1. at least one radical selected from a L- or D-α-amino acid, C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to the terminal amino group of the PTH compound, and/or at least one radical selected from C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to one or more side chain amino groups of the PTH compound,
   or
2. at least one α-amino acid unit in the positions 1 to 38 of a naturally occurring PTH sequence being replaced by a natural or unnatural amino acid unit optionally in protected form, whereby the α-amino acid units present in positions 1 and 2 at the amino terminus of the PTH sequence together may be replaced by a pseudo-peptide,
   or a combination of such modifications,
   provided that
   when the PTH compound is free from D- or L-α-amino acid attached to the N-terminus or from C₂₋₆alcoxycarbonyl or optionally substituted C₁₋₈alkyl, C₂₋₈alkenyl, C₂₋₈alkynyl, aralkyl, C₂₋₈alkynyl, aralkenyl or C₃₋₆cycloalkyl-C₁₋₄alkyl, it is other than a PTH compound having a naturally occurring α-amino acid sequence;
   or the PTH compound is other than PTH(1-34) wherein
   i. the α-amino acid in position 1 is Gly, D-Ser, D-Ala or Tyr; or
   ii. the α-amino acid in position 2 is Ala, D-Val, Lys, Arg or Cit and the α-amino acid in position 34 is Tyr; or the α-amino acid in position 2 is D-Val and the α-amino acid in position 34 is D-Tyr and optionally the α-amino acids in positions 8 and 18 are each Nle; or
   iii. the α-amino acid in positions 3 and/or 6 and/or 9 are replaced by a natural or unnatural amino acid; or
   iv. the α-amino acid in position 23 is replaced by Ala, Arg, Asn, Asp, Cys, Gln, Glu, Gly, His, Ile, Lys, Met, Pro, Ser or Thr; or
   v. the α-amino acid in position 25 and/or 26 and/or 27 is replaced by Ala, Asn, Asp, Cys, Gln, Glu, Gly, His, Ile, Leu, Met, Phe, Pro, Ser, Thr, Trp, Tyr or Val; or
   vi. the α-amino acids in positions 8 and 18 are each Nle or each Met(O) and optionally the α-amino acid in position 34 is Tyr; or the α-amino acids in positions 8 and 18 are each Nle and the α-amino acid in position 34 is Tyr and either the α-amino acid in position 12 is L- or D-pro, L- or D-Ala, Aib or NMeGly or the α-amino acid in position 23 is Phe, Leu, Nle, Val, Tyr, α-Nal or β-Nal; or
   vii. the α-amino acid in position 28 is Lys and the α-amino acid in position 30 is Leu; or
   viii. the α-amino acid in position 1 is Aib; and/or the α-amino acid in position 8 and/or 18 is Leu, Ile, Val, Phe or Trp; and/or the α-amino acid in position 11 is Ser, Lys, Phe, β-Nal, Trp or Tyr; and/or the α-amino acid in position 12 is D-Leu, D-Ile, D-Nle, D-Val, D-Ser, D-Ser(Butyl), D-Abu, D-Thr, D-Nva, D-Met, D-β-Nal, D-Trp, D-Lys, D-Tyr, D-Lys(Fmoc), D-Phe or D-Asn; and/or the α-amino acid in position 13 is Leu; and/or the α-amino acid in position 19 and/or in position 21 is Arg, Lys, Asn or His; and/or the α-amino acid in position 23 is 2-(1,3-dithiolane-2-yl)Trp; and/or the α-amino acid in position 25 and/or in position 26 is His; and/or the α-amino acid in position 27 is Gln or Leu; or
   ix. the α-amino acid in position 8 and/or 18 is Ala or Ser; or the α-amino acid in position 8 and/or 18 is Ala, Val, Leu, Ile, Ser or Trp and the α-amino acid in position 34 is Tyr; or
      the PTH compound is other than PTH(1-84) wherein
   i. the α-amino acid in position 1 is Tyr, Val, Pro, Asp or Cys; or
   ii. the α-amino acid in position 2 is Ala, Glu, Leu, Ser or Arg; or
   iii. the α-amino acid in positions 3 and/or 6 and/or 9 are replaced by a natural or unnatural amino acid; or
   iv. the α-amino acid in position 23 is replaced by Ala, Arg, Asn, Asp, Cys, Gln, Glu, Gly, His, Ile, Lys, Met, Pro, Ser or Thr; or
   v. the α-amino acid in position 25 and/or 26 and/or 27 is replaced by Ala, Asn, Asp, Cys, Gln, Glu, Gly, His, Ile, Leu, Met, Phe, Pro, Ser, Thr, Trp, Tyr or Val; or
   vi. the α-amino acid in position 8 is Met, Met(O), Ala, Val, Leu, Ile, Ser, Trp, Asn, Gln, Asp, Glu, Lys, Arg, Tyr or Gly and the α-amino acid in position 18 is Leu; or the α-amino acid in position 8 and/or 18 is Ala, Val, Leu, Ile, Ser or Trp, and optionally the α-amino acid in position 34 is Tyr; or the α-amino acid in position 8 and in position 18 are each Met(O); or the α-amino acid in position 8 is Leu and the α-amino acid in position 18 is Met(O); or
   vii. the α-amino acid in position 26 is Gln;
      the compound is other than Pro⁰PTH(1-84) or [Met⁰,Leu⁸,Leu¹⁸]PTH(1-84); or
      the PTH compound is other than hPTH(1-36) wherein the α-amino acid in position 36 is Leu;
      in free form or in salt form or complex form.

When the PTH sequence is derived from a PTH fragment, it is a PTH fragment having PTH-like activity and comprising at least the first 27 N-terminal amino acid units of PTH, preferably up to the first 38 N-terminal amino acid units of PTH, e.g. 1-34 to 1-38, e.g. 1-34, 1-36, 1-37 or 1-38 PTH, at least one of the α-amino acids being replaced according to the invention. One or more α-amino acid units normally present in the naturally occurring PTH sequence may also be omitted. hPTH fragments, particularly hPTH(1-34) and hPTH(1-36), are preferred.

The C-terminus of the PTH compounds may be —COOH, esterified —COOH, e.g. —COORₐ wherein Rₐ is lower alkyl, for example C₁₋₄alkyl, —CONH₂ or a mono- or disubstituted amide, e.g. —CONR_{b}R_{c} wherein one of R_{b} and R_{c} is H and the other is an aliphatic residue, e.g. C₁₋₆alkyl, or both are an aliphatic residue, or R_{b} and R_{c} together with the nitrogen atom to which they are attached form a heterocyclic residue, e.g. pyrrolidinyl or piperidinyl.

Hereinafter, these compounds will be referred to as compounds of the invention.

“Natural amino acids” refer to those well known in the art. They are listed and standard abbreviations are provided in the U.S.P.T.O. publication, Trademark Official Gazette, published May 15, 1990, p. 33 at 46. These amino acids and abbreviations are specifically incorporated herein by reference.

The natural amino acids are shown below:

| | | |
|---|---|---|
| A | Ala | alanine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagines |
| Q | Gln | glutamine |
| R | Arg | arginine |
| S | Ser | serine |
| T | Thr | threonine |
| V | Val | valine |
| W | Trp | tryptophane |
| Y | Tyr | tyrosine |

The term “unnatural amino acid” unit means an amino acid unit which is not genetically encoded. Examples of unnatural amino acid units include e.g. the D isomers of the natural α-amino acids as indicated above, Aib (amino-isobutyric acid), bAib (3-aminoisobutyric acid), Nva (norvaline), β-Ala, Aad (2-amino-adipic acid), bAad (3-aminoadipic acid), Abu (2-aminobutyric acid), Gaba (γ-aminobutyric acid), Acp (6-aminocaproic acid), Dbu (2,4-diaminobutyric acid), TMSA (trimethylsilyl-Ala), alle (allo-Isoleucine), Nle (Norleucine), tert.Leu, Cit (Citrulline), Orn, Dpm (2,2′-diaminopimelic acid), Dpr (2,3-diaminopropionic acid), α- or β-Nal, Cha (cyclohexyl-Ala), hydroxy-proline, Sar (Sarcosine) etc., cyclic amino acid units and N^{α}-alkylated amino acid units, e.g. MeGly (N^{α}-Methyl-glycine), EtGly (N^{α}-ethylglycine), EtAsn (N^{α}-ethyl-asparagine).

By amino acid in protected form is meant a natural or unnatural amino acid having e.g. a side chain including an heteroatom such as O, S or N which can be protected with an O-, S- or N-protecting group. The N-terminus of the PTH compounds of the invention may also be in protected form.

N-protecting groups as may be present on the N-terminus or side chain amino groups of amino acid units include such groups as e.g. disclosed in “Protective Groups in Organic Synthesis”, T. W. Greene, J. Wiley & Sons NY (1981), 219-287, for example acyl such as formyl, acetyl, trifluoroacetyl, methoxysuccinyl, hydroxysuccinyl or benzoyl optionally substituted on the phenyl ring with e.g. p-methoxycarbonyl, p-methoxy, p-nitro or p-phenylsulfonamidocarbonyl; alkoxycarbonyl such as t-butyloxycarbonyl, isobutyloxycarbonyl or methoxycarbonyl; allyloxycarbonyl; trityl; 2,2,5,7,8-pentamethyl-chroman-6-sulfonyl; arylmethoxycarbonyl such as 9-fluorenylmethoxycarbonyl or benzyloxy carbonyl optionally substituted on the phenyl ring with p-methoxy, p-nitro, o- or p-chloro, m-phenyl or 3,4-dimethyl; arylmethyl such as benzyl optionally ring substituted with p-methoxy, p-nitro or p-chloro; or arylsulfonyl such as phenyl sulfonyl optionally ring substituted with p-methyl or p-methoxy, or naphthylsulfonyl optionally ring substituted with e.g. amino or di(C₁₋₁₄alkyl)amino.

O-protecting groups of O-containing side chains are e.g. as described in “The Peptides”, 3, (1981), E. Gross and J. Meienhofer (Ed.). For aliphatic hydroxy functionalities, suitable O-protecting groups are e.g. as disclosed in the above reference in “Protection of the Hydroxy Group”, J. M. Stewart, 169-201 and include the benzyl, t.-butyl and methyl groups. For aromatic hydroxy functionalities, suitable O-protecting groups include the benzyl, t.-butyl, methyl, tosyl and benzyloxycarbonyl groups. O-protecting groups for carboxy functionalities on amino acid side chains are well known ester groups and described e.g. in “The Peptides”, 3, 101-135 (“Carboxyl Protecting Groups” by R. W. Roeske) and include the methyl, ethyl, t.-butyl and benzyl groups. S-protecting groups for thiol functionalities on amino acid side chains are known and described e.g. in “The Peptides”, 3, 137-167 (“Sulfhydryl Group Protection” by R. G. Hiskey). Examples include the methyl, t.-butyl, benzyl, p-methoxyphenylmethyl, ethylamino-carbonyl and benzyloxycarbonyl groups.

According to the invention the PTH compounds may bear on their terminal amino group at least one radical selected from a L- or D-α-amino acid, C₂₋₆alkoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl, and/or on one or more side chain amino group(s) at least one radical selected from C₂₋₆alkoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl. When such a group is attached to a side chain amino group, it is preferably on the 6-amino group of a Lys unit. Preferred substituents for the alkyl, alkenyl, aralkyl, aralkenyl or cycloalkylalkyl group are hydroxy, amino and for the aryl moiety also halogen and/or C₁₋₄alkoxy. The alkyl group or alkyl moiety may be linear or branched and optionally interrupted by O, S or N. Preferably any C₁₋₈alkyl, C₂₋₈alkenyl, aralkyl, aralkenyl or C₃₋₆cycloalkyl-C₁₋₄alkyl on the amino group of a PTH compound, is non-substituted. Examples for C₁₋₈alkyl are C₁₋₆alkyl, preferably methyl, ethyl, propyl, isopropyl, butyl, isobutyl, t.-butyl; for C₂₋₈alkenyl, C₂₋₄alkenyl, preferably allyl; for C₂₋₈alkynyl, C₂₋₄alkynyl, preferably prop-2-ynyl; for aralkyl, phenyl or benzyl; for aralkenyl, styryl; for C₃₋₆cycloalkyl-C₁₋₄alkyl, cyclohexylmethyl. C₂₋₆alkoxycarbonyl is preferably formyl or acetyl. Suitable examples of D- or L-α-amino acids attached to the N-terminus include e.g. D- or L-Pro, Ala.

Preferred substituents when present are alkyl, alkynyl, alcoxycarbonyl or a D- or L-α-amino acid attached to the N-terminus of the PTH compound and/or at least one alkyl or alkoxycarbonyl attached to one or more side chain amino groups.

Preferred PTH compounds of the invention are those comprising at least one amino acid unit replaced in one of the following positions of the PTH sequence: 1, 2, 3, 8-11, 13 to 19, 21, 22, 29 to 34, particularly 8-11, 16-19, 33 and/or 34. Further preferred PTH compounds of the invention are those comprising more than one amino acid unit replaced, particularly more than two amino acid units, more particularly more than three amino acid units, especially from 5 to 7 amino acid units replaced, preferably at any combination of the above mentioned positions of the PTH sequence.

In a series of specific or alternative embodiments, the present invention provides a PTH compound as disclosed above wherein in particular the α-amino acids in positions 1 and 2 at the amino terminus of the PTH sequence are replaced by a pseudo-dipeptide.

The term “pseudo-dipeptide” as used herein refers to a dipeptide isostere in which the peptide bond between the 2 amino acid residues, whether natural or unnatural, is replaced with any isosteric group, e.g. —CH₂—NH—, —C(Halogen)=CH— or —C (alkyl)=CH—.

Examples of compounds of the invention wherein the α-amino acids in positions 1 and 2 are replaced by a pseudo-dipeptide include e.g compounds of formula I

X—P₁ I
wherein
X is a residue of formula (a)

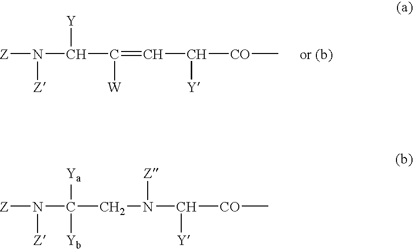

wherein each of Z and Z′, independently, is H; optionally substituted C₁₋₈alkyl, C₂₋₈alkenyl, aralkyl, aralkenyl or C₃₋₆cycloalkyl-C₁₋₄alkyl; or a protecting group, at most one of Z and Z′ being a protecting group,
Z″ is H, C₁₋₈alkyl or a protecting group
each of Y and Y′, independently, is an optionally protected side chain of a natural or unnatural α-amino acid, one of Yₐ and Y_{b} is hydrogen and the other is an optionally protected side chain of a natural or unnatural α-amino acid or Yₐ and Y_{b} form together with the carbon atom to which they are attached a C₃₋₆cycloalkyl group,
and
W is halogen or C₁₋₄alkyl,
or W and Y′ together with the moiety

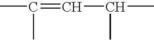

to which they are attached, form an optionally substituted aromatic cyclic or heterocyclic residue, and
   P₁ is a PTH sequence, as defined above, in which amino acid units in positions 1 and 2 of the N-terminus are omitted,
in free form or in salt or complex form.

In compounds of formula I, P₁ may be a fragment (3-z) of PTH wherein z is 84 or an integer from 27 to 38, preferably from 34 to 38, particularly 34, 36, 37 or 38, especially 34 or 36. The PTH sequence represented by P₁ may correspond either to a naturally occurring PTH sequence, or to a naturally occurring PTH sequence wherein at least one α-amino acid unit has been replaced by a natural or unnatural amino acid optionally in protected form and/or one or more α-amino acid units are also omitted. P₁ may also comprise at least one side chain amino group substituted as disclosed above.

Halogen is preferably fluorine or chlorine.

In the residue of formula (a), the double bond has preferably the configuration trans (E).

As residue attaching to the α-carbon atom of a natural α-amino acid, Y, Y′, Yₐ or Y_{b} may be the side chain present in a natural α-amino acid as listed above, e.g. as present in Gly (i.e. H), Ala, Val, Ser, Leu, Ile, Phe, Trp. Y, Y′, Yₐ or Y_{b} may also be the residue attaching to the α-carbon atom of an unnatural α-amino acid as indicated above, e.g. as present in Nva, Orn, Abu, Aib, Nle. If the side chain of the natural or unnatural amino acids as Y, Y′, Yₐ or Y_{b} includes heteroatoms such as O, S or N, the heteroatoms on the side chain may optionally be protected with an O-, S- or N-protecting group, e.g. as indicated above.

Protecting groups as Z or Z′ or Z″ may be as disclosed above. C₁₋₈alkyl as Z or Z′ or Z″ may be as indicated above.

Preferably Z or Z′ is hydrogen or C₁₋₄alkyl, particularly hydrogen or methyl.

Preferably Y is H or CH₃.

When W is C₁₋₄alkyl, it is preferably CH₃.

When W and Y′ together with the moiety to which they are attached form an optionally substituted aromatic cyclic or heterocyclic residue, it may be an aromatic 5- or 6-membered cyclic residue comprising optionally 1 or 2 heteroatoms selected from N, S and O, e.g. phenyl, imidazolyl, pyridyl, oxazolyl or thiazolyl. A preferred substituent is hydroxy or methoxy, particularly for the phenyl ring.

Y′ is preferably H, CH₃, isopropyl or benzyl.

When Z″ is C₁₋₈alkyl, it is preferably CH₃, C₂H₅ or isopropyl.

C₃₋₆cycloalkyl as Yₐ and Y_{b} together is preferably cyclopropyl or cyclopentyl.

When Z is a protecting group, it is preferably acyl, particularly acetyl.

In a series of specific and alternative embodiments, the present invention provides a PTH compound as disclosed above wherein the α-amino acid unit in position 1 and/or the α-amino acid unit in position 2 of the N-terminus of the PTH sequence is replaced by an optionally protected natural or unnatural amino acid residue, provided that when the PTH compound is free from D- or L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;

or the PTH compound is other than PTH(1-34) wherein
i. the α-amino acid in position 1 is Aib, Gly, D-Ser, D-Ala or Tyr; or
ii. the α-amino acid in position 2 is Ala, D-Val, Lys, Cit or Arg and the α-amino acid in position 34 is Tyr; or the α-amino acid in position 2 is D-Val and the α-amino acid in position 34 is D-Tyr and optionally the α-amino acids in positions 8 and 18 are each Nle; or
iii. the α-amino acid in position 1 is Aib and at least one further α-amino acid unit has been replaced as follows: the α-amino acid in position 8 and/or 18 is Leu, Ile, Val, Phe or Trp, and/or the α-amino acid in position 11 is Ser, Lys, Phe, β-Nal, Trp or Tyr, and/or the α-amino acid in position 12 is D-Leu, D-Ile, D-Nle, D-Val, D-Ser, D-Ser(Butyl), D-Abu, D-Thr, D-Nva, D-Met, D-β-Nal, D-Trp, D-Lys, D-Tyr, D-Lys(Fmoc), D-Phe or D-Asn, and/or the α-amino acid in position 13 is Leu, and/or the α-amino acid in position 19 and/or in position 21 is Arg, Lys, Asn or His, and/or the α-amino acid in position 23 is 2-(1,3-dithiolane-2-yl)Trp, and/or the α-amino acid in position 25 and/or in position 26 is His, and/or the α-amino acid in position 27 is Gln or Leu,
or the PTH compound is other than PTH (1-84) wherein
   i. the α-amino acid in position 1 is Tyr, Val, Pro, Asp or Cys; or
   ii. the α-amino acid in position 2 is Ala, Glu, Leu, Ser or Arg.

Examples of compounds of the invention wherein the α-amino acids in position 1 and/or in position 2 are replaced as indicated above include e.g. compounds of formula II

X₁ₐ—Y₂ₐ—P₂ II

wherein
X₁ₐ is a residue of an optionally protected natural α-amino acid or a residue of formula (IIa)

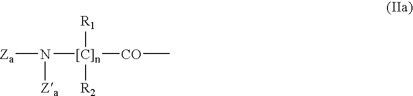

wherein
each of Zₐ and Z′ₐ, independently, is H, C₁₋₆alkyl or a protecting group, at most one of Zₐ and Z′ₐ being a protecting group, and either n is 1 and
i. each of R₁ and R₂, independently, is C₁₋₆alkyl, or
ii. one of R₁ and R₂ is methyl or ethyl and the other is an optionally protected residue attaching to the α-carbon atom of a natural α-amino acid other than Ala, Leu, Ile or Val, or
iii. one of R₁ and R₂ is H, methyl or ethyl and the other is an optionally protected residue attaching to the α-carbon atom of an unnatural α-amino acid, or
iv. R₁ and R₂ form together with the carbon atom to which they are attached a C₃₋₆cycloalkyl group, or
v. the residue

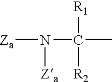

represents a heterocyclic residue optionally condensed to a benzene ring,
or n is 2, 3, 4 or 5 and each of R₁ and R₂ is H or CH₃,
Y₂ₐ is a residue of an optionally protected natural α-amino acid or a residue of formula (IIb)

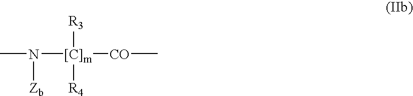

Z_{b} is H or C₁₋₆alkyl and either m is 1 and
   i. each of R₃ and R₄, independently, is C₁₋₆alkyl, or
   ii. one of R₃ and R₄ is methyl or ethyl and the other is an optionally protected residue attaching to the α-carbon atom of a natural α-amino acid other than Ala, Leu, Ile or Val, or
   iii. one of R₃ and R₄ is H, methyl or ethyl and the other is an optionally protected residue attaching to the α-carbon atom of an unnatural α-amino acid,
      or m is 2, 3, 4 or 5 and each of R₃ and R₄ is H or CH₃, and
P₂ is a PTH sequence as defined above wherein amino acid units in positions 1 and/or 2 of the N-terminus are omitted.

When the residue

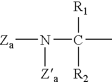

is a heterocyclic residue optionally condensed to a benzene ring, it may be a residue derived from a saturated or unsaturated 5- or 6-membered heterocycle comprising one or more heteroatoms selected from N, O and S. Examples of such heterocyclic residues include e.g. 2-pyridyl, 3-morpholinyl, hexahydropyridazinyl, 2- or 3-indolyl.

When n or m is >1, the moiety CR₁R₂ or CR₃R₄ may be —CH(CH₃)—CH₂—, propyl, butyl or pentyl.

C₃₋₆cycloalkyl as R₁ and R₂ together is preferably cyclopropyl or cyclopentyl.

As residue attaching to the α-carbon atom of a natural α-amino acid, R₁, R₂, R₃ or R₄ may be the side chain present in a natural α-amino acid as listed above, e.g. as present in Gly, Ser or Thr. One of R₁ and R₂ or one of R₃ and R₄ may be methyl or ethyl and the other may be CH₃, isopropyl, isobutyl or CH₃—CH₂—CH(CH₃)—. R₁, R₂, R₃ or R₄ may also be the residue attaching to the α-carbon atom of an unnatural α-amino acid as indicated above, e.g. as present in Abu, Gaba, Nva, Aib, TMSA or Nle. If the side chain of the natural or unnatural amino acids as R₁, R₂, R₃ or R₄ includes heteroatoms such as O, S or N, the heteroatoms on the side chain may optionally be protected with an O-, S- or N-protecting group, e.g. as indicated above. Protecting group as Zₐ or Z′ₐ may be as disclosed above. C₁₋₆alkyl as Z_{b} is preferably methyl or ethyl. Preferably Z_{b} is H. C₁₋₆alkyl as Zₐ or Z′ₐ is preferably straight chain or branched C₁₋₄alkyl. Preferably one of Zₐ and Z′ₐ is H and the other is H, C₁₋₄alkyl or a protecting group, particularly H.

In a further or alternative embodiment, the present invention also provides a PTH compound as disclosed above wherein the α-amino acid unit in position 8 and/or the α-amino acid unit in position 18 is replaced by an optionally protected natural or unnatural amino acid residue, provided that when the PTH compound is free from D- or L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;

or the PTH compound is other than PTH(1-34) wherein
i. the α-amino acids in positions 8 and 18 are each Nle or each Met(O) and optionally the α-amino acid in position 34 is Tyr; or the α-amino acids in positions 8 and 18 are each Nle and the α-amino acid in position 34 is Tyr and either the α-amino acid in position 12 is L- or D-Pro, L- or D-Ala, Aib or NMeGly or the α-amino acid in position 23 is Phe, Leu, Nle, Val, Tyr, α-Nal or β-Nal; or the α-amino acids in positions 8 and 18 are each Nle and the α-amino acid in position 2 is D-Val and the α-amino acid in position 34 is D-Tyr; or
ii. the α-amino acid in position 8 and/or 18 is Leu, Ile, Val, Phe or Trp and optionally at least one further α-amino acid unit has been replaced as follows: the α-amino acid in position 1 is Aib and/or the α-amino acid in position 11 is Ser, Lys, Phe, β-Nal, Trp or Tyr, and/or the α-amino acid in position 12 is D-Leu, D-Ile, D-Nle, D-Val, D-Ser, D-Ser(Butyl), D-Abu, D-Thr, D-Nva, D-Met, D-β-Nal, D-Trp, D-Lys, D-Tyr, D-Lys(Fmoc), D-Phe or D-Asn, and/or the α-amino acid in position 13 is Leu, and/or the α-amino acid in position 19 and/or in position 21 is Arg, Lys, Asn or His, and/or the α-amino acid in position 23 is 2-(1,3-dithiolane-2-yl)Trp, and/or the α-amino acid in position 25 and/or in position 26 is His, and/or the α-amino acid in position 27 is Gln or Leu; or
iii. the α-amino acid in position 8 and/or 18 is Ala or Ser; or the α-amino acid in position 8 and/or 18 is Leu, and the α-amino acid in position 34 is Tyr; or
   the PTH compound is other than PTH(1-84) wherein
      the α-amino acid in position 8 is Met, Met (O), Ala, Val, Leu, Ile, Ser, Trp, Asn, Gln, Asp, Glu, Lys, Arg, Tyr or Gly and the α-amino acid in position 18 is Leu; or the α-amino acid in position 8 and in position 18 are each Met(O); or the α-amino acid in position 8 is Leu and the α-amino acid in position 18 is Met(O); or
   the PTH compound is other than [Leu¹⁸,Tyr³⁴]hPTH(1-84), [Leu⁸,Tyr³⁴]hPTH(1-84), [Ile⁸,Leu¹⁸,Tyr³⁴]hPTH(1-84) or [Leu⁸, Leu¹⁸,Tyr³⁴]hPTH(1-84).

When the α-amino acid unit in position 8 is replaced by an unnatural amino acid, it may be an unnatural amino acid as disclosed above e.g. Nva, Nle or Cha. Preferably it is an unnatural lipophilic amino acid preferably such containing a straight chain alkyl residue.

Nva (norvaline) and its homologues are particularly preferred. The unnatural amino acid in position 8 of the PTH sequence may also be a Cα-methylated or Cα-ethylated natural α-amino acid.

Examples of PTH compounds of the invention wherein the α-amino acid unit in position 18 is replaced, are e.g. PTH compounds wherein the α-amino acid residue in position 18 is replaced by a natural amino acid residue such as Gln, Tyr or Lys, for example [Gln¹⁸]-PTH, [Lys¹⁸]-PTH and [Tyr¹⁸]-PTH, particularly [Gln¹⁸ or Lys¹⁸ or Tyr¹⁸]-hPTH-(1-x) wherein x is 84 or an integer from 27 to 38, particularly from 34 to 38. Ala in position 18 is also preferred particularly when the PTH sequence is a (1-36)PTH fragment. In these compounds, the amino acid in position 8 may be Met or it may be replaced by an amino acid selected from Leu, Ile, Val, Phe, Gln, Trp, Ser and Ala or it may be an unnatural lipophilic amino acid residue, e.g. Nva or Nle.

Another group of such PTH compounds of the invention comprises e.g. PTH compounds wherein the amino acid residue in position 8 is replaced by an unnatural lipophilic amino acid residue, e.g. Nle or more preferably Nva. In these compounds the amino acid in position 18 may be Met or it may be replaced by an amino acid selected from Leu, Ile, Val, Phe, Trp, Ser, Ala, Gln, Lys or Tyr, preferably Leu, Gln, Ala or Tyr.

A further group of such PTH compounds of the invention are those wherein one or more amino acid units in the remaining positions, e.g. 1 to 7, 9 to 17 and/or 19 to 38 are either omitted or replaced in addition to the replacement of the α-amino acid unit in position 8 and/or 18. In such a case, the α-amino acid in position 8 is preferably replaced by Leu and the α-amino acid in position 18 by Gln, Leu, Ala or Tyr.

In a yet further or alternative embodiment, the present invention also provides a PTH compound as disclosed above wherein at least one of the α-amino acid unit of the PTH sequence is replaced by the α-amino acid unit which is present at the corresponding position in PTHrP,

provided that
when the PTH compound is free from D- or L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;
or the PTH compound is other than PTH(1-34) wherein
i. the α-amino acid in position 8 and/or 18 is Leu; and/or the α-amino acid in position 11 is Lys; and/or the α-amino acid in position 19 and/or 21 is Arg; and/or the α-amino acid in position 25 and/or 26 is His; and/or the α-amino acid in position 27 is Leu; or
ii. the α-amino acid in position 25 in Gln and/or the α-amino acid in position 26 is Asn, the α-amino acid in position 27 being optionally Leu; or
   the PTH compound is other than [Leu⁸,Leu¹⁸]PTH(1-84).

The term “PTHrP” refers to any genetically encoded form of PTHrP, e.g. human, chicken, rat or mouse PTHrP. For consistency and as is conventional, in the following description, the same numbering system will be applied to the amino acids of the PTHrP sequence starting with Ala in position 1 and comprising e.g. Ala in position 38.

This group of compounds according to the invention are hybrids between PTH and the homologous PTHrP sequences (referred to herein as PTH-rP hybrids). The amino acid sequence wherein the substitution according to the invention takes place is from position 1 up to position 38, particularly 1 up to 36, especially 1 up to 34.

Preferred PTH compounds of the invention are PTH-rP hybrids wherein more than one α-amino acid unit of the PTH sequence, e.g. at least 2, preferably from 3 to 5 α-amino acid units, are replaced according to the invention.

A preferred group of the PTH-rP hybrids according to the invention comprises PTH compounds wherein at least one α-amino acid unit of the PTH α-amino acid sequence in positions 8 to 11, i.e. Met⁸, His⁹, Asn¹⁰ or Leu¹¹ is replaced by the corresponding α-amino acid unit of the corresponding sequence of PTHrP, i.e. Leu⁸, His⁹, Asp¹⁰ or Lys¹¹. The replacement at positions 8 and/or 10 is preferred, particularly at positions 8 and 10. More preferably the α-amino acids at positions 8, 10 and 11 of said PTH sequence are replaced by Leu⁸, Asp¹⁰ and Lys¹¹, respectively.

Another preferred group of the PTH-rP hybrids according to the invention comprises PTH compounds wherein at least one α-amino acid unit of the PTH α-amino acid sequence in positions 16 to 19 is replaced by the corresponding α-amino acid unit of the corresponding sequence of PTHrP, i.e. Gln¹⁶, Asp¹⁷, Leu¹⁸, Arg¹⁹. Preferably 3 or all 4 α-amino acids of said sequence are replaced. Particularly preferred are PTH compounds of the invention wherein the α-amino acid in position 16 is Gln, the α-amino acid in position 18 is Leu and the α-amino acid in position 19 is Arg.

A further preferred group of the PTH-rP hybrids according to the invention comprises PTH compounds wherein at least one α-amino acid unit of the PTH α-amino acid sequence in positions 33 and 34 is replaced by the corresponding α-amino acid unit of the corresponding sequence of PTHrP, i.e. Thr³³, Ala³⁴. Preferably both α-amino acids are replaced.

Yet further preferred groups of the PTH-rP hybrids according to the invention are PTH compounds comprising any combination of the above mentioned α-amino acid substitutions (8-11, 16-19, 33-34), more preferably a combination of substitutions selected from the above indicated 8-11 and 33-34 sequences.

As it will be appreciated, in the PTH-rP hybrids according to the invention one or more α-amino acids in positions 1 up to 38 may be further replaced by a natural or unnatural amino acid unit as indicated above or be omitted. In position 10 there may be a α-amino acid selected from Gly, Gln, Glu, His, Ser, Thr or Tyr. In position 13 there may be a D- or L-α-amino acid other than Arg, e.g. Ala, Cys, Gln, Ile, Asn, Trp, Asp, Val, Ser, Thr, Tyr, Met, Leu or Gly; in position 16 there may be a D- or L-α-amino, e.g. Lys, Ser, Leu, Ala, Gln or Gly; in position 17 Ala or Ser or preferably an amino acid having a bigger side chain than Ala or Ser, e.g. Glu, Gln, Phe, His, Ile or Lys; in position 18 Gln or Tyr; in position 19 Ala, Arg, Val, Tyr, Ser, Lys, Met, His, Gly, Pro, Asn or Ile; Gln or Arg in position 26; and/or in position 33 a D- or L-α-amino acid e.g. Ser, Thr, Leu, Gly, Gln, Arg, Pro, Asp, Ile, Lys, or Thr. They also may be substituted on the N-terminus or on a side chain amino group as indicated above. If desired, S- or O-containing side chains of the PTH-rP hybrids of the invention may also be protected as disclosed above.

Examples of preferred PTH-rP hybrids according to the invention are [Leu⁸, Gln¹⁸, Thr³³, Ala³⁴]PTH, [Leu⁸, Ala¹⁶, Gln¹⁸, Thr³³, Ala³⁴]PTH, [Leu⁸, Asp¹⁰, Lys¹¹, Ala¹⁶, Gln¹⁸, Thr³³, Ala³⁴]PTH, [Leu⁸, Asp¹⁰, Lys¹¹, Ala¹⁶, Gln¹⁸]PTH, [Leu⁸, Ala¹⁶, Gln¹⁸, Ala¹⁹]PTH, [Leu⁸, Asp¹⁰, Lys¹¹, Gln¹⁸]PTH, [Leu⁸, Asp¹⁰, Lys¹¹, Ala¹⁶, Gln¹⁸, Ala¹⁹]PTH, [Leu⁸, Ala¹⁶, Gln¹⁸, Ala¹⁹, Thr³³, Ala³⁴]PTH and [Leu⁸, Asp¹⁰, Lys¹¹, Gln¹⁸, Thr³³, Ala³⁴]PTH.

Preferred PTH-rP hybrids according to the invention are PTH(1-x′) compounds wherein x′ is an integer from 34 to 38, particularly 34, 36, 37 or 38 especially hPTH(1-x′), wherein one or more α-amino acids are replaced by the α-amino acid(s) present at the corresponding position in PTHrP preferably as indicated above. When the positions 33 and 34 are replaced by the corresponding α-amino acid sequence of PTHrP, the PTH compound is preferably a hPTH fragment comprising 34 α-amino acid units. PTH-rP hybrids with carboxy terminus are preferred.

Preferably PTHrP is hPTHrP.

The compounds of the invention may exist e.g. in free form, salt form or in the form of complexes thereof. Acid addition salts may be formed with e.g. organic acids, polymeric acids and inorganic acids. Such acid addition salt forms include e.g. the hydrochlorides and the acetates. Complexes are e.g. formed from the compound of the invention on addition of inorganic substances, e.g. inorganic salts or hydroxides such as Ca- and Zn-salts, and/or an addition of polymeric organic substances.

The present invention also provides a process for the production of the compounds of the invention. They may be prepared in a stepwise manner either in solution or using the solid phase synthesis process or genetic engineering.

The compounds of the invention may be produced for example as follows:
a) removing at least one protecting group which is present in a compound of the invention in protected form; or
b) linking together by an amide bond two peptide fragments, the peptide fragments being such that the desired amino acid sequence of the desired compound is obtained, and then effecting optionally stage a) of the process,
c) for the production of a PTH compound wherein the α-amino acid units in positions 1 and 2 at the amino terminus are replaced by a pseudo-dipeptide, reacting a pseudo-dipeptide in protected or unprotected form with a PTH peptide in protected or unprotected form in which the amino acid residues in positions 1 and 2 are omitted and if necessary carrying out process step a); or
d) for the production of a PTH compound comprising at least one radical selected from D- or L-α-amino acid attached to the N-terminus and C₂₋₆alkoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl attached to the terminal amino group and/or to a side chain amino group, introducing such a radical in a PTH compound in protected or unprotected form and free from such a radical, and if necessary carrying out process step a);
   and recovering the compounds thus obtained in free form, in salt form or in complex form.

Process steps a), b) and c) may be effected in analogy with known methods, e.g. as known in the art of peptide chemistry or as described in the following examples. Where desired, in these reactions, protecting groups which are suitable for use in peptides may be used for functional groups which do not participate in the reaction. The term protecting group may also include a polymer resin having functional groups.

In process step b), for the production of a PTH compound wherein the α-amino acid units in positions 1 and 2 at the amino terminus are replaced by a pseudo-dipeptide, one peptide fragment used as starting material may comprise the pseudo-dipeptide at its N-terminus. This starting material may be prepared in accordance with process step c).

In process step c) the pseudo-dipeptide used as starting material may be a compound of formula IIaa or IIbb

ZZ′N—CHY—CW═CH—CHY′—COOH (IIaa)

ZZ′N—CYₐY_{b}—CH₂—NZ″-CRY′—COOH (IIbb)

wherein Y, Y′, Yₐ, Y_{b}, Z, Z′, Z″ and W are as defined above, or a functional derivative thereof, e.g. ester, acid halide or asymetric or asymetric anhydride. Compounds of formula IIaa wherein W and Y′ together with the moiety

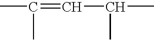

to which they are attached, form an optionally substituted aromatic cyclic or heterocyclic residue, and compounds of formula IIbb wherein Z″ is alkyl or a protecting group, e.g. acetyl, or wherein —CYₐY_{b} is C₃₋₆cycloalkyl and Z″ is H are novel and form part of the invention.

Process step d) may be carried out in analogy with known methods, e.g. alkylating methods. In a preferred embodiment of the invention, the alkylation is performed under reductive conditions, e.g. using a corresponding aldehyde or ketone. It may be performed for example in the presence of NaBH₃CN, preferably at an acidic pH, e.g. from 5 to 7. The temperature of the reaction may be e.g. from −20° C. to 100° C. It may be advantageous to carry out the reaction in an inert solvent, e.g. water, an alcohol, dioxane or DMF or a mixture thereof. A D- or L-α-amino acid may also be attached to the N-terminus in accordance with known procedure.

The peptide fragment used as starting material in process step b) may comprise one or more unnatural amino acid units; it may also be substituted on the N-terminus and/or on a side chain amino group by a radical selected from C₂₋₆alkoxycarbonyl, optionally substituted C₁₋₈alkyl, C₂₋₈alkenyl, C₂₋₈alkynyl, aralkyl, aralkenyl or C₃₋₆cycloalkyl-C₁₋₄alkyl or it may bear a D- or L-α-amino acid on the N-terminus.

The compounds of the invention or fragments thereof comprising natural α-amino acid units may also be prepared using recombinant technology.

According to a preferred embodiment of the invention there is also provided a process for the production of a medium-sized polypeptide, in which a fusion protein comprising an N-terminal bacteriophage T4 gene 55 polypeptide having the desired polypeptide linked to the C-terminal thereof, is expressed in bacterial cells.

The gene 55 polypeptide may. comprise the complete gp55 protein of bacteriophage T4 (188 amino acid residues) which is described in Gram, H. and Rüger, R; 1985; The EMBO Journal, 4, (1), pp 257-264. Alternatively a fragment, preferably an N-terminal fragment of the gp55 protein may be used. For example, a gp55 protein fragment comprising the first 112 N-terminal amino acid residues of the protein may be used. Typically, however, the gene 55 polypeptide comprises at least the first 25 N-terminal residues of the gp55 protein.

The medium-sized polypeptide may be from about 20 up to 100, e.g. 150, preferably from about 20 to about 50, amino acids in length. Examples of medium-sized polypeptides include calcitonins, endorphins, gastric inhibitory peptide(s), glucagon, neuropeptide Y, growth hormone releasing factor (GHRF), amyloid β protein fragments, hirudin, insulin, somatostatin, epidermal growth factor, nerve growth factor and PTH or fragments thereof. Particularly preferred medium-sized polypeptide which may be prepared according to the process of the present invention are the compounds of the invention or fragments thereof having natural amino acid substitutions.

Conveniently the gene 55-PTH fusion protein also comprises a cleavable linker between the gene 55 polypeptide and the desired polypeptide. Preferably the cleavable linker is chemically cleavable and more preferably contains the amino acid sequences Asp-Pro-Pro or Asn-Gly-Pro.

In a further aspect the invention provides a nucleotide sequence coding for a fusion protein comprising an N-terminal bacteriophage T4 gene 55 polypeptide and a desired medium-sized polypeptide linked to the C-terminal thereof.

The nucleotide sequence is preferably a DNA sequence suitable for expression in bacteria. The sequence typically contains nucleotides coding for a cleavable linker between the gene 55 and desired polypeptide coding sequences.

The invention also provides a bacterial expression vector comprising a DNA sequence coding for the fusion protein.

The expression vector typically contains, in addition to the fusion protein coding sequence, appropriate expression control sequences including a suitable promoter, an operator and a ribosome binding site and other appropriate regulatory sequences. Usually, also the expression vector contains one or more selectable markers.

Any suitable promoter may be used, such as the TrpE, λPl, λPr, lac or Tac promoter. Preferably the promoter is the bacteriophage T7 promoter and the expression vector is a plasmid. For E. coli expression an R1 or Col-E1 plasmid-derived vector may be used. For example, expression vector pET 17B, obtainable from Novagen, or similar expression vectors may be used.

In another aspect the invention provides bacterial host cells transformed with a fusion protein coding sequence or an expression vector as described above. Any suitable bacterial host may be used, though preferably the host is E. coli. For example, E. coli strain BL21 (DE3) Lys E and similar strains may be used.

Advantageously the fusion protein accumulates within the host cells in the form of insoluble inclusion bodies, and thereby facilitates recovery of the fusion protein product from the cells. In order to recover the desired polypeptide product from the fusion protein, the protein of the inclusion bodies is solubilised, and the desired polypeptide product cleaved from the fusion protein. Any suitable solubilisation treatment may be used, including acid treatment, e.g. at about pH 2 to 4, preferably with acid at about pH 2.5, or treatment with a denaturing agent, e.g. a mild denaturating agent such as guanidine hydrochloride. In addition it may be necessary to remove one or more unwanted N-terminal amino acid residues from the product which remain after cleavage.

Thus in a further aspect the invention provides a process for the production of a desired medium-sized polypeptide, in a bacterial host, the process comprising:

causing transformed bacterial host cells as defined above to express the fusion protein in the form of inclusion bodies;
isolating the inclusion bodies;
solubilizing the inclusion bodies, and
cleaving the desired polypeptide from the fusion protein.

In the case of some medium-sized polypeptides, such as the PTH compounds, it is not normally necessary to carry out carefully-controlled denaturation and renaturation procedures to obtain PTH products in active form, e.g. having PTH-like activity. The medium-sized polypeptide products may be obtained in active form merely by neutralising the acid conditions used for solubilisation. Solubilisation and cleavage of the fusion protein may be carried out in a single step.

The invention also includes a fusion protein comprising an N-terminal bacteriophage T4 gene 55 polypeptide and, linked to the C-terminal thereof, a desired medium-sized polypeptide.

Using the processes and vectors of the invention it has been found that it is possible to produce high levels of medium-sized polypeptides, particularly PTH products. In E. coli, we have found that up to about 50% of the total protein expressed is the desired fusion protein. Also, the fusion protein forms inclusion bodies which are easy to separate from the other material of the cells. Moreover the inclusion bodies often consist of at least about 90% of the desired fusion protein which can significantly reduce the amount of purification that is required. Further, expressing the PTH product, in the form of the fusion protein can substantially decrease endogenous processing of the polypeptide in the bacterial cell, allowing homogenous products to be obtained.

Preferably the cleavable linker contains chemically cleavable amino acids adjacent the N-terminus of the desired polypeptide, to permit the desired polypeptide to be cleaved from the fusion protein by simple chemical means. Preferably the desired polypeptide does not contain the chemically cleavable groups.

Preferred chemically cleavable linkers contain the amino acids Asp-Pro-Pro or Asn-Gly-Pro. The Asp-Pro or Asn-Gly bonds may be chemically cleaved using acid conditions e.g. formic acid (normally about pH 2.5) or hydroxylamine respectively. The dipeptides Pro-Pro or Gly-Pro that remain on the N-terminus of the desired polypeptide may then be removed using a suitable dipeptidyl-peptidase. For example the L. lactis X-Pro dipeptidyl-peptidase EC 3.4.14.5 can be used. This peptidase is described in Nardi et al; 1991; Applied and Environmental Microbiology, 57, p 45 to 50.

The desired polypeptide may be purified following cleavage from the fusion protein. HPLC purification techniques may be used. Purification may be carried out before removal of any unwanted N-terminal amino acid residues.

The process of the invention is further illustrated in the examples 306 to 312 FIG. 1 shows the amino acid sequence and corresponding DNA sequence of a truncated gp55-Asp-Pro-Pro-hPTH(1-38)OH fusion protein as cloned into the plasmid pET17B, FIG. 2 shows the amino acid sequence and corresponding DNA sequence of a full length gp55-Asn-Gly-Pro-hPTH(1-38)OH fusion protein as cloned into the plasmid pET17B.

In the following Examples all temperatures are in ° C. The following abbreviations are employed. In the present specification the term PTH without any further indication includes the carboxy terminus as well as a carboxamide terminus.

BOC=tert.Butyloxycarbonyl
DMF=dimethylformamide
DCM=dichloromethane
Fmoc=9-fluorenylmethoxycarbonyl
HOBt=1-hydroxybenzotriazole
TFA=trifluoroacetic acid
Trt=trityl=triphenylmethyl
RP-HPLC=reverse phase high performance liquid chromatography
r.t.=room temperature

### DNP=Dinitrophenyl

### Tos=Tosyl

### DIPC=Diisopropylcarbodiimide

Pmc=2,2,5,7,8-Pentamethylchroman-6-sulfonyl

### Ip=Isopropyl

### For=Formyl

Cpe=1-amino-cyclopentane-1-carboxylic acid
Cpp=1-amino-cyclopropane-1-carboxylic acid
Cha=cyclohexylalanine
tBu=t.butyl

### EXAMPLE 1

### N^{α}-Isopropyl hPTH(1-34) NH₂

This peptide is assembled in a stepwise manner on a poly-styrene-based resin support. The Boc-group is used for protection of the alpha amino-group and side-chain functional groups are protected as follows: Lys(2-chlorobenzyloxycarbonyl), Ser(benzyl), Thr(benzyl), Glu(benzyl), Asp(benzyl), Met(O), His(DNP), Arg(Tos), and Trp(HCO). Other residues are left unprotected.

Amino-4-methylphenyl-methyl-co(polystyrene-divinylbenzene=MBHA-resin) (0.7 mmol/g) is subjected to the following cycle, steps (1) to (7), of treatments:
(1) DCM
(2) trifluoracetic acid (50%) in DCM
(3) DCM
(4) diisopropylethylamine (10%) in DMF
(5) DMF
(6) preformed symmetrical anhydride (1.4 mmol per g starting resin) of Boc-amino acid in DMF
(7) DMF

Volumes of washes and reagents are from 5 to 20 ml per gram of starting resin. Each step is repeated as many times as necessary for either complete reaction of the resin (steps 2, 4, 6) or complete displacement of the previous reagent from the resin (steps 1, 3, 5, 7). Samples of resin are removed after each cycle and checked for completeness of the coupling reaction by a colorimetric test for residual amino groups using ninhydrin.

Symmetrical anhydrides of Boc-amino acids are prepared just prior to use by reacting Boc-amino acid (2.8 mmol per g resin) and DCCl (1.4 mmol per g resin) in DCM, containing DMF in amounts sufficient for complete dissolution of the Boc-amino acid. The mixture is filtered, more DMF is added to the filtrate which is concentrated by evaporation of the volatile components at a temperature not exceeding 15° C. and the resulting solution is used in step (6).

The cycle of reactions, (1) to (7), is repeated for each amino acid residue such as to provide the sequence of the title compound except for Boc-Gln-OH and Boc-Arg(Tos)-OH, which are coupled in step (6) as their preformed 1-hydroxybenzo-triazole esters in DMF.

The fully assembled peptide resin is treated twice with 20 ml of thiophenol 5% in DMF at r.t. for 1 hour and washed with DMF, methanol and dichloromethane. The peptide resin is again subjected to steps (1) to (5) followed by the addition of 0.5 ml of acetone, 84 mg of sodium cyanoborohydride and 0.2 ml of acetic acid in 20 ml of DMF per gram of resin. After 16 hours at RT the resin is washed with DMF and dichloromethane and dried.

The peptide resin is treated with liquid HF, dimethyl sulfide, p-cresol and ethane-1,2-dithiol according to the ‘low-high’ procedure which is described, e.g., in International Journal of Peptide and Protein Research, vol. 26, page 262-273 (1985). After removal of the volatile components and washing with ethylacetate the residue is extracted with several portions of 1%-acetic acid, filtered and the filtrate liophilized. The lyophilized product is purified by repeated reversed-phase chromatography on a column of octadecyl-silica using a gradient of acetonitril in 2%-phosphoric acid or in 20 mM tetramethylammonium phosphate. Fractions containing the pure compound are combined, filtered through a weakly basic ion-exchange resin in the acetate form and the filtrate lyophilized to give the title compound.

MS (Ion-Spray): 4160.

### EXAMPLE 2

### [Lys(N^{ε}-Isopropyl)^{26,27}]hPTH(1-34)NH₂

The peptide is assembled as described for Example 1 but using Lys(Fmoc) for incorporation in position 13 and Fmoc-Ser(tBu) in terminal position 1.

The fully assembled peptide resin is treated twice with 20 ml of thiophenol 5% in DMF at r.t. for 1 hour and washed with DMF, methanol and dichloromethane. The dry resin is treated with liquid HF as described for Example 1. The cleavage product (350 mg) is suspended in a mixture of methanol (5 ml), phosphate buffer pH=5.0 (5 ml), sodium cyanoborohydride (48 mg) and acetone (0.28 ml). After 16 hours at RT the reaction mixture is filtered and the filtrate evaporated. The residue is suspended in 20%-piperidine in DMF for 15 minutes, diluted with 20 volumes of ether and filtered. The residue is dissolved in 100 ml of water and acetic acid added to pH=3, filtered and the filtrate lyophilized. The lyophilizate is purified by reversed-phase chromatography on a column of octadecyl-silica using a gradient of acetonitril in 2%-phosphoric acid. Fractions containing the pure compound are combined, filtered through a weakly basic ion-exchange resin in the acetate form and the filtrate lyophilized to give the title compound.

MS (Ion Spray): 4205.6.

### EXAMPLE 3

### N^{α}-Isopropyl[Lys(N^{ε}-Isopropyl)^{13,26,27}]hPTH-(1-38)-OH

### a) Solid Phase Peptide Synthesis

The peptide is synthesized in a stepwise manner on a polystyrene based resin support. The alpha-amino group is protected by Fmoc and the side-chain functional groups as followed: Asp(OtBu), Glu(OtBu), His(Trt), Lys(Boc), Asn(Trt), Gln(Trt), Arg(Pmc) and Ser(tBu). Other amino acids are left unprotected.
Fmoc-Gly esterified to 4-Hydroxymethyl-phenoxymethyl-co(polystyrene-1%-divinylbenzene), 0.5 mmol/g, is used as starting material for the stepwise solid phase synthesis, which consists of repetitive cycles of alpha-amino deprotection, washing, coupling (attachment of new amino acid) and washing. A three to five fold excess of Fmoc-amino acids is coupled as preformed HOBt-esters using DIPC.
Fmoc-Arg(Pmc), Fmoc-Asn(Trt) and Fmoc-Gln(Trt) are coupled as symmetrical anhydrides using DIPC. After complete assembly of the peptide chain the Fmoc-protecting group of Ser¹ is removed by 20% piperidine/DMF. Cleavage of the peptide from the polystyrene resin and removal of all side chain protecting groups is obtained by treatment of the peptide resin with a mixture of 10% ethanedithiol, thioanisole, thiocresole and water in 90% TFA for three hours at room temperature. The resin particles are filtered off and washed with some TFA. The product is precipitated from the combined filtrates by addition of ether, filtered and dried. The product is purified by chromatography on a C-18 silica column using a gradient of acetonitrile in 2% H₃PO₄/water. Fractions containing the pure compound are collected, filtered through an anion-exchange resin (Biorad, AG 4-X4, 100-200 mesh acetate form) and lyophilized to give hPTH-(1-38)-OH.

### b) Alkylation

40 mg (9 μmol) hPTH-(1-38)-OH (4458.2 g/mol) are dissolved in 1.2 ml methanol, 1.2 ml phosphate buffer (Merck no. 9887 adjusted to pH 5.0), 45 mg (716 mmol) NaBH₃CN (62.84 g/mol) and 0.8 ml (11 mmol) acetone (73.52 ml/mol). The reaction is monitored by RP-HPLC on a C-18 silica column and finished after 15 hours at room temperature. The reaction mixture is diluted with water and chromatographed on a C-18 silica column using a gradient of acetonitrile in 2% H₃PO₄/water. Fractions containing the pure compound are collected, filtered through an anion-exchange resin (acetate form) and lyophilized to give the title compound as polyacetate, polyhydrate.

[α]_{D}²⁰=−5.7° (c=0.317 in 95% AcOH)

MS (Ion-Spray): 4626

### EXAMPLE 4

### N^{α}-Methyl[Ala¹]PTH-(1-38)-OH

The peptide chain is assembled in the same manner as in example 3a. At position 1 instead of Fmoc-Ser(tBu)—OH the unnatural amino acid Fmoc-Na-Methyl-Ala-OH is coupled to the peptide resin. Cleavage, deprotection and purification is performed as in example 3a.

MS (Io_n Spray)=4455.91

### EXAMPLE 5

### [Ala¹,Ala³,Leu⁸,Gln¹³,Ala¹⁶,Gln¹⁸,Ala¹⁹,Phe²³,His²⁵,His²⁶,Leu²¹,Thr³³, Ala³⁴]hPTH(1-34)OH

This peptide is prepared in analogy with the procedure of Example 3a. Instead of Fmoc-Gly esterified to 4-hydroxymethyl-phenoxy-methyl-co (polystyrene-divinylbenzene) the appropriate Fmoc-amino acid, e.g. Fmoc-Ala, Fmoc-Phe, Fmoc-D-Ala, Fmoc-D-Phe, etc. is used. Additionally the side-chain functional groups are protected as follows: Thr(t.-butyl), Trp(Boc) and Tyr(t.-butyl).

By repeating the procedure disclosed in Examples 3 and 5 but using the appropriate starting materials, the following compounds may be obtained:
EXAMPLE 6: [Leu⁸,Asp¹⁰,Ala¹⁶,Gln¹⁸,Thr³³]hPTH(1-34)OH
EXAMPLE 7: [Ile¹]hPTH(1-38)OH (IS-MS: 4484)
EXAMPLE 8: [Ala¹,Abu²]hPTH(1-38)OH (IS-MS: 4428)
EXAMPLE 9: [Ala¹,Nva²]hPTH(1-38)OH (IS-MS: 4442)
EXAMPLE 10: [Ala¹,Ile²]hPTH(1-38)OH (IS-MS: 4456)
EXAMPLE 11: [Ala¹,Ala³,Leu⁸,Gln¹³,Ala¹⁶,Gln¹⁸,Ala¹⁹,His²⁶,Leu²⁷,Thr³³,Ala³⁴]hPTH(1-34)OH
EXAMPLE 12: [N-MeAla¹]hPTH(1-36)OH (IS-MS: 4286)
EXAMPLE 13: [Ala¹,Ala³,Leu⁸,Gln¹⁸]hPTH(1-36)OH (IS-MS: 4235)
EXAMPLE 14: [Thr¹]-hPTH-(1-38)OH (IS-MS: 4472)
EXAMPLE 15: [Leu¹]-hPTH-(1-38)OH (IS-MS: 4484)
EXAMPLE 16: [Abu¹]-hPTH-(1-38)OH (IS-MS: 4456)
EXAMPLE 17: [Gaba¹]-hPTH-(1-38)OH (IS-MS: 4456)
EXAMPLE 18: [Leu⁸,Lys¹¹,Gln¹⁸]hPTH(1-36)OH
EXAMPLE 19: [Leu⁸,Gln¹⁶,Asp¹⁷,Leu¹⁸,Arg¹⁹,Arg²²]hPTH(1-36)OH (IS-MS: 4347)
EXAMPLE 20: [Leu⁸,Gln¹⁸,Thr³³,Ala³⁴]-hPTH(1-34)OH (IS-MS: 4007)
EXAMPLE 21: [Leu⁸,Ala¹⁶,Gln¹⁸,Ala¹⁹,Thr³³,Ala³⁴]hPTH(1-34)OH (IS-MS: 3906)
EXAMPLE 22: [Leu⁸,Ala¹³,Gln¹⁸,Gln²⁶,Phe²⁷,Thr³³,Ala³⁴]hPTH(1-34)OH
EXAMPLE 23: Ip-[Leu⁸,Lys(Ip)¹³,Gln¹⁸,Lys(Ip)^{26,27},Thr³³,Ala³⁴]hPTH(1-34)OH (IS-MS: 4175)
EXAMPLE 24: Ip-[Leu⁸,Ala¹³,Gln¹⁸,Gln²⁶,Phe²⁷,Thr³³,Ala³⁴]hPTH(1-34)OH
EXAMPLE 25: [Gln¹⁶]hPTH(1-38)OH
EXAMPLE 26: [Ser¹⁴]hPTH(1-38)OH
EXAMPLE 27: [Ala¹,Ala³,Leu⁸,Gln¹³,Ala¹⁶,Gln¹⁸,Ala¹⁹,His²⁵,His²⁶,Leu²⁷,Thr³³,Ala³⁴]hPTH(1-34)OH
EXAMPLE 28: [Ala¹,Ala³,Leu⁸,Gln¹³,Ala¹⁶,Gln¹⁸,Ala¹⁹,Gln²⁶,Phe²⁷,Thr³³,Ala³⁴]hPTH(3
EXAMPLE 29: [Leu⁸,Ala¹⁶,Gln¹⁸,Thr³³,Ala³⁴]hPTH(1-34)OH (IS-MS: 3965)
EXAMPLE 30: [Leu⁸,Gln¹⁸,Ala²⁹,Glu³⁰,Ile³¹]hPTH(1-34)OH
EXAMPLE 31: [Leu⁸,Asp¹⁰,Lys¹¹,Gln¹⁸]hPTH(1-36)OH
EXAMPLE 32: [Leu⁸,Asp¹⁰,Lys¹¹,Ser¹⁴,Ile¹⁵,Gln¹⁶,Asp¹⁷,Leu¹⁸,Arg¹⁹]hPTH(1-36)OH
EXAMPLE 33: [Leu⁸,Asp¹⁰,Lys¹¹,Leu¹⁸]hPTH(1-36)OH
EXAMPLE 34: [Leu⁸,Gln¹⁶,Asp¹⁷,Leu¹⁸,Arg¹⁹]hPTH(1-36)OH
EXAMPLE 35: [Leu⁸,Asp¹⁰,Lys¹¹,Ala¹⁷,Leu¹⁸]hPTH(1-36)OH (IS-MS: 4252)
EXAMPLE 36: [Leu⁸,Gln¹⁶,Asp¹⁷,Leu¹⁸,Arg¹⁹,Thr³³,Ala³⁴]hPTH(1-34)OH
EXAMPLE 37: [Leu⁸,Asp¹⁰,Lys¹¹,Gln¹⁸,Thr³³,Ala³⁴]hPTH(1-34)OH (IS-MS: 4022)
EXAMPLE 38: [Leu⁸,Ala¹⁶,Asp¹⁷,Leu¹⁸,Ala¹⁹]hPTH(1-36)OH
EXAMPLE 39: [Leu⁸,Asp¹⁰,Ala¹⁶,Asp⁷,Leu¹⁸,Ala¹⁹]hPTH(1-36)OH
EXAMPLE 40: [Leu⁸,Gln¹⁸,Arg²²,Thr³³,Ala³⁴]hPTH(1-34)OH
EXAMPLE 41: [Leu⁸,Asp¹⁰,Lys¹¹,Gln¹⁶,Asp⁷,Leu¹⁸,Arg¹⁹,Thr³³,Ala³⁴]hPTH(1-34)OH (IS-MS: 4077)
EXAMPLE 42: [Leu⁸,Asp¹⁰,Lys¹¹,Ala¹⁶,Gln¹⁸,Ala¹⁹]hPTH(1-36)OH (IS-MS: 4181)
EXAMPLE 43: [Leu⁸,Ala¹⁶,Asp¹⁷,Gln¹⁸,Ala¹⁹]hPTH(1-36)OH (IS-MS: 4193)
EXAMPLE 44: [Leu⁸,Ala¹⁶,Ala¹⁷,Gln¹⁸,Ala¹⁹]hPTH(1-36)OH (IS-MS: 4149)
EXAMPLE 45: [Leu⁸,Ala¹⁷,Gln¹⁸,Ala¹⁹]hPTH(1-36)OH
EXAMPLE 46: [Leu⁸,Ala¹⁷,Gln¹⁸,Ala¹⁹,Arg²²]hPTH(1-36)OH (IS-MS: 4219)
EXAMPLE 47: [Leu⁸,Ala¹⁷,Gln¹⁸,Ala¹⁹,Arg²²,Thr³³,Ala³⁴]hPTH(1-34)OH (IS-MS: 3960)
EXAMPLE 48: [Leu⁸,Gln¹⁸]hPTH(1-36)OH
EXAMPLE 49: [Leu⁸,Asp¹⁰,Lys¹¹,Ala¹⁶,Gln¹⁸,Thr³³,Ala³⁴]hPTH(1-34)OH (IS-MS: 3980)
EXAMPLE 50: [Leu⁸,Asp¹⁰,Lys¹¹,Ala¹⁶,Gln¹⁸]hPTH(1-36)OH (IS-MS: 4240)
EXAMPLE 51: [Leu⁸,Asp¹⁰,Lys¹¹,Ala¹⁶,Gln¹⁸,Ala¹⁹,Thr³³,Ala³⁴]hPTH(1-34)OH (IS-MS: 3923)
EXAMPLE 52: [Leu⁸,Asp¹⁰,Ala¹⁶,Gln¹⁸]hPTH(1-36)OH (IS-MS: 4225)
EXAMPLE 53: [Leu⁸,Asp¹⁰,Lys¹¹,Ala¹⁶,Gln¹⁸,Ala¹⁹,Thr³³]hPTH(1-34)OH (IS-MS: 3999)
EXAMPLE 54: [Leu⁸,Gln¹³,Ala¹⁶,Gln¹⁸,Ala¹⁹,His²⁶,Leu²⁷,Thr³³]hPTH(1-34)OH
EXAMPLE 55: [Leu⁸,Ala¹⁶,Gln¹⁸,Ala¹⁹,Arg²²]hPTH(1-36)OH
EXAMPLE 56: [Ile¹⁵]hPTH(1-38)OH
EXAMPLE 57: [Leu⁸,Gln¹³,Ala¹⁶,Gln¹⁸,Ala¹⁹,Arg²²,His²⁶,Leu²⁷,Thr³³,Ala³⁴]hPTH(1-34)OH
EXAMPLE 58: [Leu⁸,Gln¹³,Ala¹⁶,Gln¹⁸,Arg¹⁹,His²⁶,Leu²⁷,Thr³³,Ala³⁴]hPTH(1-34)OH
EXAMPLE 59: [Leu⁸,Ser¹³,Ala¹⁶,Gln¹⁸,Ala¹⁹,Arg²²]hPTH(1-36)OH
EXAMPLE 60: [Leu⁸,Ala¹³,Ala¹⁶,Gln¹⁸,Ala¹⁹,Arg²⁶,Arg²⁷]hPTH(1-36)OH
EXAMPLE 61: [Leu⁸,Gln¹³,Ala¹⁶,Gln¹⁸,Ala¹⁹,His²⁶,Leu²⁷,Arg³³,Ala³⁴]hPTH(1-34)OH
EXAMPLE 62: [Leu⁸,Ala^{16,17,18,19}]hPTH(1-36)OH
EXAMPLE 63: [Leu⁸,Ala¹³,Ala¹⁶,Gln¹⁸,Ala¹⁹,Gln²⁶,Phe²⁷]hPTH(1-36)OH (IS-MS: 4127)
EXAMPLE 64: Ip-[Leu⁸,Ala¹³,Ala¹⁶,Gln¹⁸,Ala¹⁹,Gln²⁶,Phe²⁷]hPTH(1-36)OH
EXAMPLE 65: Ip-[Leu⁸,Lys(Ip)¹³,Ala¹⁶,Gln¹⁸,Ala¹⁹,Lys(Ip)^{26,27}]hPTH(1-36)OH
EXAMPLE 66: [Leu⁸,Ala¹⁶,Gln¹⁸,Ala¹⁹]hPTH(1-36)OH (IS-MS: 4166)
EXAMPLE 67: Ip-[Leu⁸,Lys(Ip)¹³,Ala¹⁶,Ala¹⁷,Ala¹⁸,Ala¹⁹,Lys(Ip)^{26,27}]hPTH(1-36)OH
EXAMPLE 68: [Aib³,Gln¹⁸]hPTH(1-36)OH (IS-MS: 4283)
EXAMPLE 69: Ip-[Leu⁸,Asp¹⁰,Lys(Ip)^{11,13,26,27},Gln¹⁸]hPTH(1-36)OH
EXAMPLE 70: Ip-[Leu⁸,Ser¹³,Ala¹⁶,Gln¹⁸,Ala¹⁹,Arg²²,Lys(Ip)^{26,27}]hPTH(1-36)OH
EXAMPLE 71: [Leu⁸,Tyr¹⁸]hPTH(1-36)OH
EXAMPLE 72: [Ser³³]-hPTH-(1-38)-OH
EXAMPLE 73: [Thr³³]-hPTH-(1-38)-OH
EXAMPLE 74: [Leu³³]-hPTH-(1-38)-OH
EXAMPLE 75: [Gly³³]-hPTH-(1-38)-OH
EXAMPLE 76: [Leu⁸,His¹⁰,Gln¹⁸,Arg²²,Thr³³,Ala³⁴]hPTH(1-34)OH
EXAMPLE 77: [Leu⁸,Gly¹⁰,Gln¹⁸,Arg²²,Thr³³,Ala³⁴]hPTH(1-34)OH
EXAMPLE 78: [Leu⁸,Glu¹⁰,Gln¹⁸,Arg²²,Thr³³,Ala³⁴]hPTH(1-34)OH
EXAMPLE 79: [Leu⁸,Thr¹⁰,Gln¹⁸,Arg²²,Thr³³,Ala³⁴]hPTH(1-34)OH
EXAMPLE 80: [Leu⁸,Gln¹⁰,Gln¹⁸,Arg²²,Thr³³,Ala³⁴]hPTH(1-34)OH
EXAMPLE 81: [Gln³³]-hPTH-(1-38)-OH
EXAMPLE 82: [Leu⁸,Tyr¹⁰,Gln¹⁸,Arg²²,Thr³³,Ala³⁴]hPTH(1-34)OH
EXAMPLE 83: [1-amino-cyclopentane-1-carboxylic acid¹,Leu⁸,Ala¹⁶,Gln¹⁸,Ala¹⁹]hPTH(1-36)OH
EXAMPLE 84: [1-amino-cyclopentane-1-carboxylic acid¹,Leu⁸,Ala^{13,16},Gln¹⁸,Ala¹⁹,Arg^{26,27}]hPTH(1-36)OH
EXAMPLE 85: [1-amino-cyclopentane-1-carboxylic acid³,Gln¹⁸]hPTH(1-36)OH (IS-MS 4309)
EXAMPLE 86: [Arg¹²]-hPTH-(1-38)-OH
EXAMPLE 87: [Ser¹²]-hPTH-(1-38)-OH
EXAMPLE 88: [Cys¹³]-hPTH-(1-38)-OH
EXAMPLE 89: [Ile¹³]-hPTH-(1-38)-OH
EXAMPLE 90: [Asn¹³]-hPTH-(1-38)-OH
EXAMPLE 91: [Trp¹³]-hPTH-(1-38)-OH
EXAMPLE 92: [Asp¹³]-hPTH-(1-38)-OH
EXAMPLE 93: [Val¹³]-hPTH-(1-38)-OH
EXAMPLE 94: [Thr¹³]-hPTH-(1-38)-OH
EXAMPLE 95: [Ser¹³]-hPTH-(1-38)-OH
EXAMPLE 96: [Tyr¹³]-hPTH-(1-38)-OH
EXAMPLE 97: [Met¹³]-hPTH-(1-38)-OH
EXAMPLE 98: [Gln¹³]-hPTH-(1-38)-OH
EXAMPLE 99: [Leu¹³]-hPTH-(1-38)-OH
EXAMPLE 100: [Ala¹³]-hPTH-(1-38)-OH
EXAMPLE 101: [Gly¹³]-hPTH-(1-38)-OH
EXAMPLE 102: [Val¹⁴]-hPTH-(1-38)-OH
EXAMPLE 103: [Ala¹⁴]-hPTH-(1-38)-OH
EXAMPLE 104: [Lys¹⁴]-hPTH-(1-38)-OH
EXAMPLE 105: [Arg¹⁴]-hPTH-(1-38)-OH
EXAMPLE 106: [Thr¹⁴]-hPTH-(1-38)-OH
EXAMPLE 107: [Ile¹⁴]-hPTH-(1-38)-OH
EXAMPLE 108: [Tyr¹⁴]-hPTH-(1-38)-OH
EXAMPLE 109: [Tyr¹⁵]-hPTH-(1-38)-OH
EXAMPLE 110: [Arg¹⁵]-hPTH-(1-38)-OH
EXAMPLE 111: [Val¹⁵]-hPTH-(1-38)-OH
EXAMPLE 112: [Lys¹⁶]-hPTH-(1-38)-OH
EXAMPLE 113: [Ser¹⁶]-hPTH-(1-38)-OH
EXAMPLE 114: [Leu¹⁶]-hPTH-(1-38)-OH
EXAMPLE 115: [Ala¹⁶]-hPTH-(1-38)-OH
EXAMPLE 116: [Gly¹⁶]-hPTH-(1-38)-OH
EXAMPLE 117: [Ala¹⁷]-hPTH-(1-38)-OH
EXAMPLE 118: [Met¹⁷]-hPTH-(1-38)-OH
EXAMPLE 119: [Ile¹⁷]-hPTH-(1-38)-OH
EXAMPLE 120: [Ser¹⁹]-hPTH-(1-38)-OH
EXAMPLE 121: [Lys¹⁹]-hPTH-(1-38)-OH
EXAMPLE 122: [Leu¹⁹]-hPTH-(1-38)-OH
EXAMPLE 123: [Ala¹⁹]-hPTH-(1-38)-OH
EXAMPLE 124: [Tyr¹⁹]-hPTH-(1-38)-OH
EXAMPLE 125: [Met¹⁹]-hPTH-(1-38)-OH
EXAMPLE 126: [His¹⁹]-hPTH-(1-38)-OH
EXAMPLE 127: [Val¹⁹]-hPTH-(1-38)-OH
EXAMPLE 128: [Gly¹⁹]-hPTH-(1-38)-OH
EXAMPLE 129: [Pro¹⁹]-hPTH-(1-38)-OH
EXAMPLE 130: [Asp¹⁹]-hPTH-(1-38)-OH
EXAMPLE 131: [Ile¹⁹]-hPTH-(1-38)-OH
EXAMPLE 132: [Val¹⁹,Gln²⁴]-hPTH-(1-38)-OH
EXAMPLE 133: [Arg¹⁹]-hPTH-(1-38)-OH
EXAMPLE 134: [Phe²⁰]-hPTH-(1-38)-OH
EXAMPLE 135: [Ala²]-hPTH-(1-38)-OH
EXAMPLE 136: [Gly²¹]-hPTH-(1-38)-OH
EXAMPLE 137: [Phe²¹]-hPTH-(1-38)-OH
EXAMPLE 138: [Leu²¹]-hPTH-(1-38)-OH
EXAMPLE 139: [Asn²¹]-hPTH-(1-38)-OH
EXAMPLE 140: [Gln²¹]-hPTH-(1-38)-OH
EXAMPLE 141: [Ser²¹]-hPTH-(1-38)-OH
EXAMPLE 142: [Gly²²]-hPTH-(1-38)-OH
EXAMPLE 143: [Leu²²]-hPTH-(1-38)-OH
EXAMPLE 144: [His²²]-hPTH-(1-38)-OH
EXAMPLE 145: [Ala²²]-hPTH-(1-38)-OH
EXAMPLE 146: [Ile²²]-hPTH-(1-38)-OH
EXAMPLE 147: [Val²²]-hPTH-(1-38)-OH
EXAMPLE 148: [Ser²²]-hPTH-(1-38)-OH
EXAMPLE 149: [Arg²²]-hPTH-(1-38)-OH
EXAMPLE 150: [Arg²⁶]-hPTH-(1-38)-OH
EXAMPLE 151: [Val²⁷]-hPTH-(1-38)-OH
EXAMPLE 152: [Ile²⁷]-hPTH-(1-38)-OH
EXAMPLE 153: [Leu²⁷]-hPTH-(1-38)-OH
EXAMPLE 154: [Arg²⁷]-hPTH-(1-38)-OH
EXAMPLE 155: [Ala²⁷]-hPTH-(1-38)-OH
EXAMPLE 156: [Val²⁸]-hPTH-(1-38)-OH
EXAMPLE 157: [Ile²⁸]-hPTH-(1-38)-OH
EXAMPLE 158: [Pro³,Thr³³]-hPTH-(1-38)-OH
EXAMPLE 159: [Arg³³]-hPTH-(1-38)-OH
EXAMPLE 160: [Pro³³]-hPTH-(1-38)-OH
EXAMPLE 161: [Asp³³]-hPTH-(1-38)-OH
EXAMPLE 162: [Ile³³]-hPTH-(1-38)-OH
EXAMPLE 163: [Lys³³]-hPTH-(1-38)-OH
EXAMPLE 164: [Ile³¹,Arg³³]-hPTH-(1-38)-OH

### EXAMPLE 165

### [1-aminocyclopentane-1-carboxylic acid1]hPTH-(1-36)NH₂

The peptide is assembled in a stepwise manner on a polystyrene based resin support. The Fmoc-group is used for protection of the alpha-amino groups.

Side-chain functional groups are protected as Arg(Pmc), ASn(Trt), Asp(OtBu), Gln(Trt), Glu(OtBu), His(Trt), Lys(Boc), Ser(tBu), Thr (tBu), Trp (Boc) and Tyr (tBu). Other amino acids are left unprotected.

4-(2′,4′-Dimethoxyphenyl-Fmoc-amino-methyl)-phenoxy-co(polystyrene-divinylbenzene), 0.4 mmol/g, which may be prepared, e.g., as described in Tetrah. Letters, 28, 3787-3790 (1987) is subjected to the following cycle, steps (1) to (5), of treatments:
(1) DMF
(2) piperidine (20%) in DMF
(3) DMF
(4) mixture of HOBt, diisopropylcarbodiimide, and Fmoc-alanine (0.8 mmol per gram starting resin each)
(5) DMF

Volumes of washes and reagents are from 5 to 20 mL per gram of starting resin.

In the next cycle of treatments (1) to (5), Fmoc-valine is substituted for Fmoc-alanine and so on for each cycle such as to assemble on the resin the correct amino acid sequence of the title compound.

Each step is repeated as many times as necessary for either complete reaction of the resin (steps 2, 4) or complete displacement of the previous reagent(s) from the resin (steps 3, 5). Samples of the resin are removed after each cycle and checked for completeness of the coupling reaction by a calorimetric test for residual amino groups using ninhydrin.

At the end of the synthesis a final cycle comprising steps (1) to (3) only is performed and the peptide resin is washed with 2-propanol, then with a mixture of methanol and methylene chloride (1:1 v/v) and dried throughly in a vacuum desiccator.

The peptide resin (1 g) is suspended in a mixture (20 ml) of trifluoroacetic acid, water, and 1,2-ethanedithiol (90:5:5 v/v) for 2 hours at room temperature, the resin particles are filtered off and washed with a small volume of TFA. The product is precipitated from the combined filtrates by addition of ether (20 volumes), filtered, washed with more ether and dried. The residue is dissolved in 2% acetic acid, the solution let to stand at r.t. for 8 hours prior to lyophilization. The lyophilisate is chromatographed on a C-18 silica column using a gradient of acetonitrile in 2% H₃PO₄. Fractions are checked by analytical HPLC and those containing the pure compound are collected, filtered through an anion-exchange resin in the acetate form and lyophilised to give the title compound as a polyacetate, polyhydrate.

Fmoc-1-aminocyclopentane-1-carboxylic acid used in the preparation of the peptide resin intermediate may be prepared, e.g. as described by G. Valle et al., 1988, in Can. J. Chem. 66:2575-2582.

MS (Ion-Spray): 4312

### EXAMPLE 166

### [1-(1-aminocyclopropane-1-carboxylic acid)]hPTH-(1-36)NH₂

This peptide is prepared in analogy with the procedure of Example 165. Fmoc-1-aminocyclopropane-1-carboxylic acid used in the preparation of the peptide resin intermediate may be prepared, e.g., as described by M. Crisma et al. (1989) in Int. J. Biol. Macromol. 11:345-352. MS (Ion-Spray): 4283

By repeating the procedure as disclosed in Example 165 but using the appropriate starting materials, following compounds may be obtained:
EXAMPLE 167: [D-Pro¹]hPTH(1-36)NH₂
EXAMPLE 168: [Nva¹]hPTH(1-36)NH₂
EXAMPLE 169: [N-Me-Ser¹]hPTH(1-36)NH₂
EXAMPLE 170: [Indole-2-carboxylic acid¹]hPTH(1-36)NH₂
EXAMPLE 171: [Indole-3-carboxylic acid¹]hPTH(1-36)NH₂
EXAMPLE 172: [Pyridine-3-carboxylic acid¹]hPTH(1-36)NH₂
EXAMPLE 173: [Pyridine-2-carboxylic acid¹]hPTH(1-36)NH₂
EXAMPLE 174: [Hexahydropyridazine-3-carboxylic acid¹]hPTH(1-36)NH₂
EXAMPLE 175: [Morpholine-2-carboxylic acid¹]hPTH(1-36)NH₂
EXAMPLE 176: [Pro¹]hPTH(1-36)NH₂
EXAMPLE 177: [Leu¹]hPTH(1-36)NH₂
EXAMPLE 178: [Ile¹]hPTH(1-36)NH₂
EXAMPLE 179: [Thr³³,Ala³⁴]hPTH(1-34)NH₂
EXAMPLE 180: [Nva⁸]hPTH(1-36)NH₂
EXAMPLE 181: [Gln¹⁸]hPTH(1-36)NH₂
EXAMPLE 182: [Tyr¹⁸]hPTH(1-36)NH₂
EXAMPLE 183: [Lys¹⁸]hPTH(1-36)NH₂
EXAMPLE 184: [Ala¹⁸]hPTH(1-36)NH₂
EXAMPLE 185: [Phe²³,His²⁵,His²⁶,Leu²⁷]-hPTH(1-34)NH₂
EXAMPLE 186: [Phe²³]-hPTH(1-36)NH₂ (IS-MS: 4247)
EXAMPLE 187: [Phe²³,His²⁵,His²⁶,Leu²⁷,Ile²⁸,Ala²⁹,Glu³⁰,Ile³¹,Thr³³,Ala³⁴]-hPTH(1-34)NH₂ (IS-MS: 3934)
EXAMPLE 188: [Ala²⁹]-hPTH(1-36)NH₂ (IS-MS: 4248)
EXAMPLE 189: [Ala³⁴]-hPTH(1-36)NH₂ (IS-MS: 4210)
EXAMPLE 190: [Gln²⁵]hPTH(1-36)NH₂
EXAMPLE 191: [Leu⁸,Asp¹⁰,Lys¹¹,Thr³³,Ala³⁴]hPTH(1-34)NH₂
EXAMPLE 192: [Ala¹,His⁵,Leu⁸,Asp¹⁰,Lys¹¹,Thr³³,Ala³⁴]hPTH(1-34)NH₂
EXAMPLE 193: [Ser¹⁴,Ile¹⁵,Gln¹⁶,Asp¹⁷,Leu¹⁸,Arg¹⁹,Thr³³,Ala³⁴]hPTH(1-34)NH₂
EXAMPLE 194: [D-Phe³⁴, D-Ala³⁶]hPTH(1-36)-NH₂ [MS(IS): 4290]
EXAMPLE 195: [Ala³]hPTH(1-36)NH₂ [MS(IS): 4271]
EXAMPLE 196: [D-Ser³]hPTH(1-36)NH₂ [MS(IS): 4290]
EXAMPLE 197: [D-Glu⁴]hPTH(1-36)NH₂ [MS(IS): 4290]
EXAMPLE 198: [D-His⁹]hPTH(1-36)NH₂ [MS(IS): 4286]
EXAMPLE 199: [Ala¹⁰]hPTH(1-36)NH₂ [MS(IS): 4243]
EXAMPLE 200: [D-Asn¹⁰]hPTH(1-36)NH₂ [MS(IS): 4288]
EXAMPLE 201: [Ala¹²]hPTH(1-36)NH₂ [MS(IS): 4299]
EXAMPLE 202: [Gln¹³]hPTH(1-36)NH₂ [MS(IS): 4287]
EXAMPLE 203: [His¹³]hPTH(1-36)NH₂ [MS(IS): 4296]
EXAMPLE 204: [Leu¹³]hPTH(1-36)NH₂ [MS(IS): 4371]
EXAMPLE 205: [Ala¹³]hPTH(1-36)NH₂ [MS(IS): 4228]
EXAMPLE 206: [Ala¹³,Gln²⁶,Phe²⁷ D-Phe³⁴ D-Ala³⁶]hPTH(1-36)NH₂ [MS(IS): 4249]
EXAMPLE 207: [Ala¹⁴]hPTH(1-36)NH₂ [MS(IS): 4219]
EXAMPLE 208: [D-His¹⁴]hPTH(1-36)NH₂ [MS(IS): 4288]
EXAMPLE 209: [D-Asn¹⁶]hPTH(1-36)NH₂ [MS(IS): 4284]
EXAMPLE 210: [Ala¹⁷hPTH(1-36)NH₂ [MS(IS): 4270]
EXAMPLE 211: [D-Ser¹⁷]hPTH(1-36)NH₂ [MS(IS): 4288]
EXAMPLE 212: [Ala¹⁹]hPTH(1-36)NH₂ [MS(IS): 4228]
EXAMPLE 213: [D-Glu¹⁹]hPTH(1-36)NH₂ [MS(IS): 4288]
EXAMPLE 214: [Ala²¹]hPTH(1-36)NH₂ [MS(IS): 4257]
EXAMPLE 215: [Ala²²]hPTH(1-36)NH₂ [MS(IS): 4227]
EXAMPLE 216: [Gln²⁶]hPTH(1-36)NH₂ [MS(IS): 4287]
EXAMPLE 217: [Nle²⁶]hPTH(1-36)NH₂ [MS(IS): 4271]
EXAMPLE 218: [D-Lys²⁶]hPTH(1-36)NH₂ [MS(IS): 4288]
EXAMPLE 219: [Nle^{8,18,27}]hPTH(1-36)NH₂
EXAMPLE 220: [His²⁷]hPTH(1-36)NH₂ [MS(IS): 4294]
EXAMPLE 221: [Phe²⁷]hPTH(1-36)NH₂ [MS(IS): 4304]
EXAMPLE 222: [Nle²⁷]hPTH(1-36)NH₂ [MS(IS): 4271]
EXAMPLE 223: [Asn²⁷]hPTH(1-36)NH₂ [MS(IS): 4271]
EXAMPLE 224: [Ala²⁷]hPTH(1-36)NH₂ [MS(IS): 4228]
EXAMPLE 225: [D-Gln²⁹]hPTH(1-36)NH₂ [MS(IS): 4289]
EXAMPLE 226: [D-Asp³⁰]hPTH(1-34)NH₂ [MS(IS): 4118]
EXAMPLE 227: [Ala³⁰]hPTH(1-36)NH₂ [MS(IS): 4241]
EXAMPLE 228: [D-Val³¹]hPTH(1-36)NH₂ [MS(IS): 4290]
EXAMPLE 229: [Ala³¹]hPTH(1-36)NH₂ [MS(IS): 4258]
EXAMPLE 230: [Lys³²]hPTH(1-34)NH₂ [MS(IS): 3832]
EXAMPLE 231: [D-His³²]hPTH(1-36)NH₂ [MS(IS): 4288]
EXAMPLE 232: [Ala³²]hPTH(1-36)NH₂ [MS(IS): 4219]
EXAMPLE 233: [D-Asn³³]hPTH(1-36)NH₂ [MS(IS): 4288]
EXAMPLE 234: [Ala³³]hPTH(1-36)NH₂ [MS(IS): 4210]
EXAMPLE 235: [NMePhe³⁴]hPTH(1-36)NH₂ [MS(IS): 4301]
EXAMPLE 236: [D-Asp³⁰]hPTH(1-36)NH₂ [MS(IS): 4290]
EXAMPLE 237: Ip-[Nle^{8,18},Lys(Ip)^{13,26,27},D-Asn³³ D-Phe³⁴]hPTH(1-34)NH₂
EXAMPLE 238: Ip-[Nle^{8,18,27},Lys(Ip)^{13,26}]hPTH(1-36)NH₂
EXAMPLE 239: [Nle^{8,18},D-Asn³³ D-Phe³⁴]hPTH(1-34)NH₂ [MS(IS): 4080]
EXAMPLE 240: [Lys(Ip)¹³]hPTH(1-36)NH₂ [MS(IS): 4331]
EXAMPLE 241: Propargyl-[A¹]-hPTH-(1-36)-NH₂
EXAMPLE 242: [Ala⁰]-hPTH-(1-36)-NH₂
EXAMPLE 243: [Pro⁰]-hPTH-(1-36)-NH₂
EXAMPLE 244: Acetyl-hPTH-(1-36)-NH₂
EXAMPLE 245: [HyPro¹]-hPTH-(1-36)-NH₂
EXAMPLE 246: N-Dimethyl-[Ala¹]-hPTH-(1-36)-NH₂
EXAMPLE 247: [D-Ser¹]-hPTH-(1-36)-NH₂ [MS(IS): 4288]
EXAMPLE 248: [Lys(For)¹]-hPTH-(1-36)-NH₂ [MS(IS): 4356]
EXAMPLE 249: [D-Glyceric acid¹]-hPTH-(1-36)-NH₂
EXAMPLE 250: [Asn¹]-hPTH-(1-36)-NH₂
EXAMPLE 251: [4-Aminobenzoic acid¹]-hPTH-(1-36)-NH₂
EXAMPLE 252: [4-Aminosalicylic acid¹]-hPTH-(1-36)-NH₂
EXAMPLE 253: [TMSA¹]-hPTH-(1-36)-NH₂ [MS(IS): 4343]
EXAMPLE 254: [Phe¹]-hPTH-(1-36)-NH₂
EXAMPLE 255: [Propargylglycin¹]-hPTH-(1-36)-NH₂
EXAMPLE 256: [Ala¹,His⁵,Leu⁸,Asp¹⁰,Lys¹¹,Gln¹⁸,Phe²²,Phe²³,His²⁵,His²⁶,Leu²⁷,Thr³³,Ala³⁴]-hPTH-(1-34)-NH₂
EXAMPLE 257: [Abu²]-hPTH-(1-36)-NH₂
EXAMPLE 258: [D-Val²]-hPTH-(1-36)-NH₂ [MS(IS): 4288]
EXAMPLE 259: [tert.Leu²]-hPTH-(1-36)-NH₂
EXAMPLE 260: [Ala¹]-hPTH-(1-36)-NH₂
EXAMPLE 261: [D-Ile5]-hPTH-(1-36)-NH₂ [MS(IS): 4288]
EXAMPLE 262: [D-Gln⁶]-hPTH-(1-36)-NH₂ [MS(IS): 4288]
EXAMPLE 263: [D-Leu⁷]-hPTH-(1-36)-NH₂ [MS(IS): 4288]
EXAMPLE 264: [Nle¹]-hPTH-(1-36)-NH₂ [MS(IS): 4268]
EXAMPLE 265: [Phe⁸]-hPTH-(1-36)-NH₂ [MS(IS): 4303]
EXAMPLE 266: [Cha⁸]-hPTH-(1-36)-NH₂ [MS(IS): 4309]
EXAMPLE 267: [Leu⁸]-hPTH-(1-38)-NH₂
EXAMPLE 268: [D-Leu¹¹]-hPTH-(1-36)-NH₂ [MS(IS): 4287]
EXAMPLE 269: [Ala¹¹]-hPTH-(1-36)-NH₂ [MS(IS): 4244]
EXAMPLE 270: [D-Lys¹³]-hPTH-(1-36)-NH₂ [MS(IS): 4288]
EXAMPLE 271: [D-Leu¹⁵]-hPTH-(1-36)-NH₂ [MS(IS): 4288]
EXAMPLE 272: [Ala¹⁵]-hPTH-(1-36)-NH₂ [MS(IS): 4243]
EXAMPLE 273: [Ala¹⁶]-hPTH-(1-36)-NH₂ [MS(IS): 4243]
EXAMPLE 274: [Met(O₂)¹⁸]-hPTH-(1-36)-NH₂ [MS(IS): 4320]
EXAMPLE 275: [Nle¹⁸]-hPTH-(1-36)-NH₂ [MS(IS): 4268]
EXAMPLE 276: [D-Met¹⁸]-hPTH-(1-36)-NH₂ [MS(IS): 4288]
EXAMPLE 277: [Lys²⁰]-hPTH-(1-36)-NH₂ [MS(IS): 4259]
EXAMPLE 278: [D-Arg²⁰]-hPTH-(1-36)-NH₂ [MS(IS): 4288]
EXAMPLE 279: [D-Val²¹]-hPTH-(1-36)-NH₂ [MS(IS): 4288]
EXAMPLE 280: [Trp(SO₂Pmc)²³]-hPTH-(1-38)-NH₂ [MS(IS): 4723]
EXAMPLE 281: [Trp(Pmc)²³]-hPTH-(1-38)-NH₂ [MS(IS): 4660]
EXAMPLE 282: [D-Trp²³]-hPTH-(1-36)-NH₂ [MS(IS): 4288]
EXAMPLE 283: [Ala²³]-hPTH-(1-36)-NH₂ [MS(IS): 4171]
EXAMPLE 284: [D-Leu²⁴]-hPTH-(1-36)-NH₂ [MS(IS): 4288]
EXAMPLE 285: [Phe²⁵]-hPTH-(1-36)-NH₂ [MS(IS): 4277]
EXAMPLE 286: [Lys²⁵]-hPTH-(1-36)-NH₂ [MS(IS): 4258]
EXAMPLE 287: [Ala²⁵]-hPTH-(1-36)-NH₂ [MS(IS): 4201]
EXAMPLE 288: [Ala²⁶]-hPTH-(1-36)-NH₂ [MS(IS): 4229]
EXAMPLE 289: [Lys²⁶¹²⁷(For)]-hPTH-(1-34)-NH₂
EXAMPLE 290: [D-Lys²⁷]-hPTH-(1-36)-NH₂ [MS(IS): 4288]
EXAMPLE 291: [D-Leu²⁸]-hPTH-(1-36)-NH₂ [MS(IS): 4288]
EXAMPLE 292: [D-Phe³⁴]-hPTH-(1-36)-NH₂ [MS(IS): 4288]
EXAMPLE 293: [D-Val³⁵]-hPTH-(1-36)-NH₂
EXAMPLE 294: [Ala³⁵]-hPTH-(1-36)-NH₂
EXAMPLE 295: [Pro³⁵]-hPTH-(1-36)-NH₂
EXAMPLE 296: [NMeVal³⁵]-hPTH-(1-36)-NH₂
EXAMPLE 297: [Thr³⁵,Ala³⁶]-hPTH-(1-36)-NH₂
EXAMPLE 298: [D-Ala³⁶]-hPTH-(1-36)-NH₂
EXAMPLE 299: [NMeAla³⁶]-hPTH-(1-36)-NH₂

### EXAMPLE 300

### (5-Amino-4-fluoro-2-isopropyl)-hex-3-enoyl-hPTH(3-36)NH₂

### a) Preparation of hPTH-(3-36)-NH-resin

This intermediate is prepared in analogy with the procedure of Example 165.

### b) [(5-Amino-4-fluoro-2-isopropyl)-hex-3-enoyl]hPTH(3-36)-amide

204.6 mg Fmoc-hPTH(3-36)-NH-resin obtained in a) above are treated with DMF for 5 min. and then washed several times with isopropanol and DMF. The Fmoc-protecting group is removed by treatment with DMF/piperidin 8:2 for 10 min. The deprotected PTH-fragment, 38.1 mg of 4-fluoro-2-isopropyl-5-t.-butoxycarbonylamino-hex-3-enoic acid, 68.7 mg benzotriazol-1-yloxytris(pyrrolidino)phosphonium hexafluorophosphate (PyBOP) and 26.6 mg 4-methylmorpholin are shaken for 80 min. in 0.7 ml DMF. After several washings with isopropanol and DMF, the solid residue is treated with 4.5 ml TFA/0.25 ml water/0.25 ml ethanedithiol for 55 min. This mixture is then filtered and by addition of diethyl ether the title compound precipitated from the remaining solution. The title compound is purified by prep.

HPLC (Vydac column, gradient A=water, B=7 acetonitril/3 water/0.2 phosphoric acid).

MS (Ion-spray): mass 4272

[α]_{365 nm}^{Hg}=−66.8° (c=0.25 in 95% AcOH)

### EXAMPLE 301

### 4-Fluoro-2-isopropyl-5-tert.butoxycarbonylamino-hex-3-enoate

### a) (S)-3-(1-Oxo(4-fluoro-3-hydroxy-2-isopropyl)hex-4-enyl)-4-pheny1 methyl-2-oxazolidinone

The title compound is prepared analogous to the procedure described by D. A. Evans et al., Org. Synth. 68, 1990; starting from (S)-3-(1-oxoisobutyl)-4-phenylmethyl-2-oxazolidinone and 2-fluoro-but-2-enal.

MS (FAB): MH⁺ 350

### b) (S)-3-(1-Oxo(4-fluoro-3-O-(2,2,2-trichlorethanimino)-2-isopropyl)hex-4-enyl)-4-phenylmethyl-2-oxazolidinone

1.5 g of the compound of Example 301a) are dissolved in 6 ml DCM. At 0° 0.0958 ml 1,8-diazabicyclo(5,4,0)undec-7-en (DBU) are added. To this solution 0.475 ml trichloroacetonitril in 2 ml DCM is added dropwise at 0°. After 1 hour the reaction mixture is evaporated and the residue is purified by column chromatography on silica gel (hexan/diethyl ether 2:1).

MS (FAB): MH⁺ 493

### c) (S)-3-(1-Oxo(4-fluoro-5-N-2,2,2-trichloracetylamino-2-isopropyl)hex-3-enyl)-4-phenylmethyl-2-oxazolidinone

2 g of the compound of 301b) is dissolved in 150 ml o-xylene and stirred under reflux at 1600 for 3 hours. Then o-xylene is removed and the residue is purified by column chromatography on silica gel (toluol/ethyl acetate 98:2).

MS (FAB): MH+ 493

### d) 4-Fluoro-5-N-2,2,2-trichloracetylamino-2-isopropyl-hex-3-enoic acid

The title compound is prepared analogous to the procedure described by D. A. Evans et al. Org. Synth. 68, 1990, but using as starting material the compound of 301c).

MS (FAB): MH⁺ 335

### e) 5-Amino-4-fluoro-2-isopropyl-hex-3-enoic acid

899 mg of the compound of 301d) are dissolved in 20 ml ethanol and 13.5 ml 6N sodium hydroxide solution are added. This mixture is stirred for 20 hours at r.t. Then ethanol is removed and the remaining aqueous fraction is acidified with 2N HCl to pH=2 and extracted with n-butanol. The extract is evaporated and the residue is filtered through silica gel (DCM/methanol 1:1), thus yielding pure title compound.

MS (FAB): MH⁺ 190

### f) 4-Fluoro-2-isopropyl-5-tert.butoxycarbonylamino-hex-3-enoic acid

0.56 g of the compound of 301e) are dissolved in 12 ml water and 1.5 g sodium carbonate are added. To this solution 0.4 g di-tert.butylcarbonate dissolved in 12 ml tetrahydrofuran are added. This mixture is stirred at room temperature for 18 hours. Then 50 ml n-butanol and 50 ml water are added and under vigorous stirring 1N HCl is added until pH=2. The organic fraction is evaporated and the residue purified by column chromatography on silica gel (hexan/acetone 1:2).

MS (FAB): MH⁺ 290

[α]_{D}=−61.3° (1% in methanol)

By repeating the above procedures but using the appropriate starting materials the following compounds may be prepared:
4-Fluoro-2-methyl-5-tert.butoxycarbonylamino-hex-3-enoic acid
4-Chloro-2-methyl-5-tert.butoxycarbonylamino-hex-3-enoic acid
4-Fluoro-2-benzyl-5-tert.butoxycarbonylamino-hex-3-enoic acid
4-Chloro-2-isopropyl-5-tert.butoxycarbonylamino-hex-3-enoic acid
4-Methyl-2-isopropyl-5-tert.butoxycarbonylamino-hex-3-enoic acid

### EXAMPLE 302

By following the procedure of Example 300 above but using the appropriate starting materials, the following compounds may be prepared:

(5-Amino-4-fluoro-2-methyl)-hex-3-enoyl-h PTH (3-36)-amide MS (Ion-Spray): Mass 4245

(5-Amino-4-chloro-2-methyl)-hex-3-enoyl-hPTH(3-36)-amide MS (Ion-Spray): Mass 4260

(5-Amino-4-fluoro-2-benzyl)-hex-3-enoyl-hPTH (3-36)-amide MS (Ion-Spray): Mass 4320

(5-Amino-4-chloro-2-isopropyl)-hex-3-enoyl-hPTH(3-36)-amide MS (Ion-Spray): Mass 4289

(5-Amino-4-methyl-2-isopropyl)-hex-3-enoyl-hPTH(3-36)-amide MS (Ion-Spray): Mass 4269

### EXAMPLE 303

By following the procedure of Example 300 above but using the appropriate starting materials, the following compounds may be prepared:

a) (5-amino-3-aza-2-isopropyl)-hexanoyl-hPTH(3-36)-amide [MS (Ion-Spray): 4257] using as starting material: (4-aza-2-t.-butoxycarbonylamino-5-isopropyl)-hexanoic acid

b) (5-amino-3-aza-3-N-acetyl-2-isopropyl)-hexanoyl-hPTH (3-36)-amide [MS (Ion-Spray): 4299] using as starting material: (4-aza-4-N-acetyl-2-t.-butoxycarbonylamino-5-isopropyl)-hexanoic acid

c) (5-amino-3-aza-3-N-acetyl)-hexanoyl-hPTH(3-36)-amide [MS (Ion-Spray): 4257] using as starting material: (4-aza-4-N-acetyl-2-t.-butoxycarbonylamino)-hexanoic acid

d) (5-methylamino-3-aza-2-isopropyl)-hexanoyl-hPTH (3-36)-amide [MS (Ion-Spray): 4271] using as starting material: [4-aza-2-(N-t.-butoxycarbonyl,N-methylamino)-5-isopropyl]-hexanoic acid

e) (5-methylamino-3-aza-3-N-methyl-2-isopropyl)-hexanoyl-hPTH(3-36)-amide [MS (Ion-Spray): 4285] using as starting material: [4-aza-4-N-methyl-2-(N-t.-butoxycarbonylamino, N-methyl-amino)-5-isopropyl]-hexanoic acid

f) (5-amino-3-aza-3-N-methyl-2-isopropyl)-hexanoyl-hPTH (3-36)-amide [MS (Ion-Spray): 4271] using as starting material: (4-aza-4-N-methyl-2-t.-butoxycarbonylamino-5-isopropyl)-hexanoic acid

g) (5-amino-3-aza-3-N-isopropyl)-hexanoyl-hPTH(3-36)-amide [MS (Ion-Spray): 4257] using as starting material: (4-aza-4-N-isopropyl-2-t.-butoxycarbonylamino-5-isopropyl)-hexanoic acid

h) [1-cyclopentane-1-amino-1-carboxylic acid (psi CH₂—NH)-Val²]-hPTH(1-36)-amide using 1-cyclopentane-1-t.-butoxycarbonyl-amino-1-carboxylic acid (psi CH₂—NH)-Val-OH as starting material.

### EXAMPLE 304

By following the procedure of Example 3a) above but using the appropriate starting material, the following compound may be prepared:

(5-amino-3-aza-2-isopropyl)-hexanoyl-[Leu⁸,Ala¹⁶,Gln¹⁸,Ala¹⁹]hPTH(3-36)OH [MS (Ion-Spray): 4135] using as starting material: (4-aza-2-t.-butoxycarbonylamino)-hexanoic acid

### EXAMPLE 305

### (4-Aza-4-N-acetyl-2-tert.butoxycarbonylamino-5-isopropyl)-hexanoic acid

### a) Methyl(4-aza-2-tert.butoxycarbonylamino-5-isopropyl)-hexanoic acid

Boc-Ala-Val-OMe is treated with the Lawesson-reagent (S.-O. Lawesson, et al. Tetrahedron 1981, 37, 3635). The resulting endothiopeptide is then reduced in accordance with the procedure described by F. S. Guziec et. al. THL, 1990, 23-26.

MS (EI): MH⁺ 289

### b) Methyl(4-aza-4-N-acetyl-2-tert.butoxycarbonylamino-5-isopropyl)hexanoate

264 mg of the compound of 305a) is dissolved in 5 ml DCM and then 0.14 ml triethylamin and 0.072 ml acetyl chloride are added. This mixture is stirred for 24 hours at 40°. After addition of 50 ml DCM, the solution is washed twice with water. The organic fraction is dried and evaporated. The title compound thus obtained is purified by column chromatography (hexan/ethyl acetate 4:1)

MS (EI): M⁺ 330

### c) (4-Aza-4-N-acetyl-2-tert.butoxycarbonylamino-5-isopropyl)-hexanoic acid

356 mg of the compound of 305b) are dissolved in 4 ml methanol/-water 3:1 and 53 mg lithium hydroxide are added. This mixture is stirred at 60° for two hours. Then 25 ml DCM are added and 1N HCl is added under vigorous stirring until pH=2. The organic fraction is separated, dried and evaporated, thus yielding the title compound in pure form.

MS (FAB): MH⁺ 317

By repeating the procedure but using the appropriate starting materials, following compounds may be obtained:
(4-Aza-4-N-acetyl-2-tert.butoxycarbonylamino)-hexanoic acid
(4-Aza-2-tert.butoxycarbonylamino-5-isopropyl)-hexanoic acid
[4-aza-2-(N-t.-butoxycarbonyl,N-methylamino)-5-isopropyl]-hexanoic acid
(4-aza-4-N-methyl-2-t.-butoxycarbonylamino-5-isopropyl)-hexanoic acid
(4-aza-4-N-isopropyl-2-t.-butoxycarbonylamino-5-isopropyl)-hexanoic acid
(4-aza-2-t.-butoxycarbonylamino)-hexanoic acid

The compounds of Examples 1 to 300 and 302 to 304 show correct amino acid ratios in quantitative amino acid analysis.

In the following examples illustrating the recombinant process of the invention, if not otherwise specified, the standard procedures described in Ausubel et al; 1991; Current Protocols in Molecular Biology, John Wiley & Sons, New York are used.

### EXAMPLE 306

### Cloning Bacteriophage T4 Gene 55

Two DNA fragments, one containing the entire coding sequence of bacteriophage T4 gene 55, and the other fragment encoding part of it are amplified by polymerase chain reaction (PCR) using purified T4 DNA as the template. Oligonucleotides which contain NdeI or BamHI endonuclease recognition sequences are used as primers. The PCR reactions (25 cycles, each 30″ 94° C., 30″ 50° C., 30″ 72° C.) are performed with 1 ng of template and 100 pM of the upstream primer (GAGGTGCATA TGTCAGAAAC TAAGCCT 27), the NdeI recognition site being provided by nucleotides 7-12 thereof) and 100 pM of the downstream primer (GGTCGGATCC ATCGTTAGCG TTAGCCTCAT ATAAAAAATC 40) the BamHI, recognition site being provided by nucleotides 5-10 thereof) in 100 μl reaction buffer containing 10 mM Tris-HCl, 50 mM KCl, 1.5 mM MgCl₂, 0.2 mM of each of dATP, dCTP, dGTP, dTTP, and 0.1% Triton X 100, pH 9.0. A 0.6 kb fragment is obtained.

The truncated form of gene 55 is prepared by PCR as described for the entire gene, except that a different oligonucleotide is used as downstream primer (GGTCGGATCC TACGTTGGAC GAATGCAT 28, the BamHI recognition site being provided by nucleotides 5-10 thereof). A 0.35 kb fragment is obtained.

The 0.6 kb and 0.35 kb DNA fragments are digested with restriction endonucleases NdeI/BamHI and are cloned into a NdeI/BamHI digest of expression vector pET17B (obtained from Novagen). The 0.6 kb DNA fragment encodes the entire gp55 protein (188 amino acid residues—see FIG. 1), whereas the 0.35 kb fragment encodes a 12 kD amino-terminal fragment of gp55 (112 amino acid residues—see FIG. 2).

### EXAMPLE 307

### Preparation and Cloning of the DNA Encoding PTH (1-38)

DNA fragments encoding a) the linker peptide corresponding to amino acid residues 110-115 of FIG. 1 and PTH (1-38) or b) the linker peptide corresponding to amino acid residues 186-191 of FIG. 2 and PTH (1-38) are generated by PCR using a cloned synthetic PTH (1-38) coding sequence as a template. The oligonucleotides encoding the linker a) (GAGTGGATCC ATCGATCCAC CATCCGTATC AGAAATACAA CT 42), or encoding linker b GAGTGGATCC GTTAACGGTC CATCCGTATC AGAAATACAA CT 42 are used as upstream primers. The downstream primer is TCTACTCGAG TTAACCCAGA GCTACAAAAT TATG 34

The upstream primers each incorporate a BamHI cloning site and the downstream primer contains a XhoI recognition site (nucleotides 5-10 of each sequence). The PCR reaction is performed under the same conditions as described in Example 306. After cleavage with BamHI and XhoI, the 0.14 kb PCR products are inserted into BamHI-XhoI digests of the gp55-pET17B plasmids, obtained in Example 306.

For expression of the fusion proteins the resultant plasmid constructs are transformed into E. coli BL21 (DE3) LysE and single bacterial colonies are further propagated. The expression of the fusion proteins is obtained by incubating a preculture at 37° C. on a rotary shaker overnight in circle growth medium (Bio 101). A main culture is inoculated with the preculture making up 1 to 5% of the volume of the main culture. The main culture is then fermented at a temperature of 37° C. and at a pH of 6.9 to 7.1 whilst being aerated at 10 l/min and agitated at 200-400 rpm.

Expression is induced by the addition of 1 mM IPTG as soon as the culture reaches an OD₅₅₀ of about 1.0 to 5.0. After induction the fermentation is continued for five hours and then the fermentation broth is harvested without killing the E. coli cells. The harvested cells are spun down with a tubular centrifuge and resuspended in sample buffer and assayed for expression by SDS PAGE. The cell pellet is then frozen until purification. The procedures and host cells used are fully described in Studier et al; 1990; “Use of T7 RNA Polymerase to direct expression of Cloned Genes”, Methods in Enzymology, Academic Press, pages 60 to 89.

### EXAMPLE 308

### Preparation of Inclusion Bodies

Frozen E. coli pellets obtained from Example 307 are resuspended to 25% w/v in Buffer A (50 mM Tris pH 8.0; containing 2 mM DTT, 5 mM Benzamidine-HCl, 1.5 mM MgCl₂, 1.0 mM MnCl₂, 10 μg/ml DNAse 1 and 2 mg/ml Lysozyme) and mixed by stirring for 1 hour at room temperature. The resuspended cells are lysed by passage through a Manton-Gaulin homogeniser (2 passes at 1200 bar) and the resultant lysate kept on ice. The lysate is diluted 2 fold with ice-cold buffer B (50 mM Tris pH 8.0; containing 2 mM DTT, 5 mM Benzamidine-HCl and 4 mM EDTA) and centrifuged for 30 min at 27,500 g.

The supernatant from the centrifuge is carefully removed and discarded. The pellet is resuspended in buffer B to 25% w/v, passed once more through the Manton-Gaulin and centrifuged as before. This process is repeated once using buffer B and once using water. The resultant inclusion body pellets are weighed and frozen at −20° C. until required.

### EXAMPLE 309

### Solubilisation and Cleavage of gp55-Asp-Pro-Pro-(1-38)hPTH Inclusion Bodies

Frozen inclusion bodies containing gp55-Asp-Pro-Pro-(1-38)hPTH as obtained in Example 308 are suspended in 10 mM HCl acid solution (analytical grade) to a final concentration of 6% w/v. The solution is incubated for 24 hours at 50° C. and the reaction terminated by addition of 1 volume of aqueous 100 mM NaOAc solution. The diluted supernatant is clarified by centrifugation at 27,500 g for 30 minutes and filtered through a glass fibre filter.

### EXAMPLE 310

### Solubilisation and Cleavage of gp55-Asn-Gly-Pro-(1-38)hPTH Inclusion Bodies

Frozen inclusion bodies containing gp55-Asn-Gly-Pro-(1-38)hPTH and obtained from example 308 are dissolved in 6M Guanidine-HCl (containing 2M Hydroxylamine-HCl and brought to pH 9.0 by addition of 4.5M LiOH) to give an estimated 5 mg/ml protein. The pH is readjusted to pH 9 by further addition of LiOH and the solution incubated for 4 hours at 45° C. The reaction is terminated by addition of formic acid to pH 4 and the solution centrifuged and filtered as described in example 309.

### EXAMPLE 311

### Preparative HPLC of Gly-Pro and Pro-Pro (1-38)hPTH

Filtered cleavage supernatants are analysed on analytical reversed phase HPLC (Orpogen HD-Gel-RP-7s-300; 4×150 mm column) and compared with a known (1-38)hPTH standard (Bachem) to determine the PTH concentration. The column is equilibrated with 85% HPLC buffer A (90% water, 10% acetonitrile; containing 0.1% v/v TFA) and 15% HPLC buffer B (10% water, 90% acetonitrile; containing 0.1% TFA) and is eluted with a gradient of 15% HPLC buffer B to 100% HPLC buffer B over 15 minutes at a flow rate of 0.75 ml/min.

Depending upon the scale of the preparation, supernatants are either loaded on a 1.0×25 cm C4 Vydac or a 2.2×25 cm C4 Vydac. The columns are equilibrated with 85% HPLC buffer A and 15% HPLC buffer B at flow rates of 4 ml/min and 10 ml/min respectively. The columns are eluted with gradients of 15% B to 85% B over 80 ml and 15% B to 55% B over 300 ml respectively.

Gly-Pro-hPTH(1-38) or Pro-Pro-hPTH(1-38) peaks are collected manually and the acetonitrile is later removed under vacuum. Analytical HPLC is used to determine the peptide concentration both after cleavage and in the collected fractions.

The solvent free peptide solutions are diluted with an equal volume of 100 mM sodium acetate and the pH adjusted, if necessary, to pH 5.4. Following filtration (0.2 μm), the solution is loaded on a cation exchange column (either Mono S or SP-Sepharose High Performance, packed into HR10/10 or XK16/10 columns respectively) equilibrated with 50 mM sodium acetate pH 5.4 (buffer C). The column is eluted with a 0 to 500 mM NaCl gradient in buffer C over 7.5 column volumes. 10 ml fractions are collected and the X—X-(1-38)hPTH peak is pooled. The peptide concentration is determined as before using HPLC.

This column serves both to exchange the peptides into a more physiological buffer and to remove additional peptides produced by chemical cleavage of the fusion protein itself.

### EXAMPLE 312

### Enzymatic Removal of X—X from X—X-(1-38)hPTH

Pooled peptide fractions, as obtained in Example 311, (concentration range 1.5-2.0 mg/ml) are brought to pH 8.0 by addition of solid Tris. Purified X-Prolyl dipeptidase (0.5 mg/ml in pas pH 7.2) (Nardi et al. (1991) App. Env. Microbiol. 57, No. 1, 45-50) is added to 1:1000 and the solution is incubated for 24 h at 37 C. The reaction is stopped by addition of TFA to 0.2% (pH=5.0) and the resultant (1-38)hPTH is isolated using preparative HPLC (using the same conditions as described in Example 311). The solvent is removed and the product is lyophilised after removal of aliquots for N-terminal sequence and mass spectroscopic analyses.

Examples of the Yields which are Obtained Using the Two Fusion Protein Approaches Described Above are as Follows:

### Gen55-Asp-Pro-Pro-(1-38)PTH (Using the “Truncated” Form of Gen55)

Under partially optimized fermentation conditions, a 215 g wet cell pellet is obtained from a 10 litre fermentation. The percentage level of expression of the fusion protein is approximately 37%. From this pellet, 31 g of inclusion bodies are isolated. The protein purity (with respect to the fusion protein) of these bodies is 61%. After solubilisation, cleavage and centrifugation/filtration approximately 620 mg Pro-Pro-PTH are detected. This material yields 580 mg Pro-Pro-PTH after subsequent C4-Vydac HPLC. After dilution, cation-exchange on Mono S, digestion with X-Prolydipeptidypeptidase (at a Pro-Pro-PTH concentration of 2 mg/ml), HPLC and lyophilisation, 440 mg of lyophilized pure product with full biological activity is obtained. This represents a yield of 71% between the stopped cleavage step and the final product.

### Gen55-Asn-Gly-Pro-(1-38)PTH (Using the “Long” Form of Gen55)

From a non-optimized 20 litre fermentation a 78 g wet cell pellet with 50% expression of the fusion protein is harvested. Following lysis and centrifugation, 18 g of “90% pure” inclusion bodies are obtained. Following the acid stop step and centrifugation/filtration about 470 mg Gly-pro-PTH are detected. After HPLC on C4-Vydac, 355 mg of peptide are detected, decreasing to 321 mg after SP-Sepharose cation-exchange. Enzymatic removal of the Gly and Pro residues is carried out at 2 mg/ml. After the final HPLC step and lyophilization, 244 mg of pure, fully biologically active (1-38)PTH is recovered representing a yield of 52% from the stopped cleavage reaction stage.

Compounds of Examples 25, 26, 56 and 86 to 164 are also prepared using the process of Examples 306 to 312.

The compounds of the invention in free form or in the form of pharmaceutically acceptable salts and complexes exhibit valuable pharmacological properties as indicated in animal tests and are therefore indicated for therapy.

The biological activity of the compounds of the invention is assessed in vitro by measuring their ability of stimulating the synthesis of cyclic AMP in UMR 106-06 rat and SaOS-2 human osteosarcoma cells according to the method of Marcus and Aurbach in Endocrinology, 85, 801-810 (1969). Rat osteosarcoma UMR 106 cells are grown to confluence in Eagle's Minimum Essential Medium −10% FCS in 12 well plates, human SaOS-2 osteosarcoma cells are grown in McCoy's SA medium −10% FCS. The medium is then changed to medium with 2% FCS and 1-5 μCi/well [3H]-adenine is added. Two hours later, cells are washed twice with serum-free medium and incubated in DMEM −1% BSA containing 1 mM 3-isobutyl-1-methylxanthine to exclude actions on phosphodiesterases. Test substances are added 20 min later. The reaction is stopped and cAMP extracted after 15 min by adding ice cold 5% trichloroacetic acid. A carrier solution (0.5 ml/well) containing 5 mM of unlabelled adenine, adenosine, AMP, ADP, ATP, and cAMP as well as 0.4 μCi [14C]-adenosine for determination of recovery is added. [3H]-cAMP is separated using serial Dowex 50W-X4 (200-400 mesh) and alumina chromatography and counted. Results are calculated in % of solvent control and EC₅₀ values determined from DRC curves. Compounds of the invention stimulate cAMP production in UMR 106-06 rat and SaOS-2 human at a concentration of 10-11 to 10-6 M. Compounds of Examples 29, 37 and 49 have an EC₅₀ value in the UMR 106-06 cells of 8.96, 8.98 and 9.00 nM, respectively.

The compounds of the invention also have binding affinity to PTH receptors, e.g. as follows:

Chicken [Tyr³⁶]PTHrP(1-36)amide is iodinated to a specific activity of 2,200 Ci/mmol using the lactoperoxidase method (Anawa Lab. AG, Wangen). Monolayers of opossum kidney cells are washed with 200 μl DMEM and HAM's F12 (1:1) containing 1% BSA and incubated at 16° C. with 50.000 cpm of [¹²⁵I-Tyr³⁶]chPTHrP(1-36)-amide per well in the presence or absence of 1 μM [Tyr³⁶]chPTHrP-(1-36) amide. After incubation, cells are washed with 0.5 ml medium (4° C.), lysed with 0.5 ml 1N NaOH and radioactivity is determined. Specific binding is defined as total binding minus nonspecific binding. Competition curves are analyzed using SCTFIT, a non-linear regression computer program (Feyen et al, 1992, Biochem. Biophys. Res. Commun. 187:8-13) and data presented as mean pK_{D} values (n=2 to 3). Compounds of the invention show in this test a binding affinity expressed as a mean pK_{D} value of from 7 to 10. Compounds of Examples 29, 37 and 49 have a pK_{D} value of 8.97, 9.13 and 8.99, respectively.

Furthermore, the compounds of the invention increase plasma calcium level after continuous s.c. infusion, e.g. as determined in male thyroparathyroidectomized Wistar rats weighing 140 to 170 g. 5 days after thyroparathyroidectomy, rats are implanted in the neck with Alzet minipumps and test compounds infused at rates of 0.3 to 30 μg/day/rat. Blood is drawn by retro-orbital puncture in the morning of days 1, 2, 3, and 6 thereafter and plasma calcium concentrations are determined photometrically.

The compounds of the invention increase bone mass in old rats after treatment for 4 weeks at a dosage range of from 50 to 800 μg/kg/day administered s.c. once daily.

The compounds of the invention are accordingly indicated for preventing or treating all bone conditions which are associated with increased calcium depletion or resorption or in which calcium fixation in the bone is desirable, e.g. osteoporosis of various genesis (e.g. juvenile, menopausal, post-menopausal, post-traumatic, caused by old age or by cortico-steroid therapy or inactivity), fractures, osteopathy, including acute and chronic states associated with skeletal demineralisation, osteo-malacia, periodontal bone loss or bone loss due to arthritis or osteoarthritis, or for treating hypoparathyroidism.

The compounds of the invention are particularly indicated for preventing or treating osteoporosis of various genesis.

For these indications, the appropriate dosage will, of course, vary depending upon, for example, the host, the mode of administration and the severity of the conditions being treated. However, in general, satisfactory results in animals are indicated to be obtained at daily dosages from about 0.0001 to about 0.5 mg/kg animal body weight. In larger mammals, for example humans, an indicated daily dosage is in the range from about 0.003 to about 10 mg, preferably 0.003 to 3, more preferably 0.01 to 1 mg, of the compounds of the invention. Compounds of the invention may be administered once a day or up to twice a week.

The compounds of the invention may be administered in free form or in pharmaceutically acceptable salt form or complexes. Such salts and complexes may be prepared in conventional manner and exhibit the same order of activity as the free compounds. The present invention also provides a pharmaceutical composition comprising a compound of the invention in free base form or in pharmaceutically acceptable salt form or complex form in association with a pharmaceutically acceptable diluent or carrier. Such compositions may be formulated in conventional manner. The compounds of the invention may be administered by any conventional route, for example parenterally e.g. in form of injectable solutions or suspensions, enterally, e.g. orally, for example in the form of tablets or capsules or in a transdermal, nasal or a suppository form.

In accordance with the foregoing the present invention further provides:
a) a compound of the invention or a pharmaceutically acceptable salt or complex thereof for use as a pharmaceutical;
b) a method for improving bone formation, e.g. preventing or treating all bone conditions which are associated with increased calcium depletion or resorption or in which calcium fixation in the bone is desirable, e.g. osteoporosis of various genesis (e.g. juvenile, menopausal, post-menopausal, post-traumatic, caused by old age or by cortico-steroid therapy or inactivity), fractures, osteopathy, including acute and chronic states associated with skeletal demineralisation, osteo-malacia, periodontal bone loss or bone loss due to arthritis or osteoarthritis, or for treating hypoparathyroidism, in a subject in need of such treatment, which method comprises administering to said subject an effective amount of a compound of the invention or a pharmaceutically acceptable salt or complex thereof.

According to a further embodiment of the invention, the compounds of the invention may be employed as adjunct or adjuvant to other therapy, e.g. a therapy using a bone resorption inhibitor, for example as in osteoporosis therapy, in particular a therapy employing calcium, a calcitonin or an analogue or derivative thereof, e.g. salmon, eel or human calcitonin, a steroid hormone, e.g. an estrogen, a partial estrogen agonist or estrogen-gestagen combination, a biphosphonate, vitamin D or an analog thereof, or any combination thereof.

When the compounds of the invention are administered in conjunction with, e.g. as an adjuvant to bone resorption inhibition therapy, dosages for the co-administered inhibitor will of course vary depending on the type of inhibitor drug employed, e.g. whether it is a steroid or a calcitonin, on the condition to be treated, whether it is a curative or preventive therapy, on the regimen and so forth.

In accordance with the foregoing the present invention provides in a yet further aspect:
d) a method for improving bone formation, e.g. preventing or treating calcium depletion, for example for preventing or treating any of the specific conditions or diseases hereinbefore set forth, in a subject in need of such a treatment which method comprises administering to said subject an effective amount of a) a compound of the invention and b) a second drug substance, said second drug substance being a bone resorption inhibitor, for example as indicated above.

Compounds of Examples 20, 29, 37, 49 and 50 are preferred. It is indicated that these compounds may be administered at a dosage of from 0.01 to 1 mg s.c. to larger mammals, for example humans, once a day or up to twice a week.

1. A PTH compound having PTH-like activity and comprising at least one modification, said modification being either
1. at least one radical selected from a L- or D-α-amino acid, C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to the terminal group of the PTH compound, and/or at least one radical selected from C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₁₄alkyl and attached to one or more chain amino groups of the PTH compound,
or
2. at least one amino acid unit in the positions 1 to 38 of a naturally occurring PTH sequence being replaced by a natural or unnatural amino acid unit optionally in protected form, whereby the α-amino acid units present in positions 1 and 2 at the amino terminus of the PTH sequence together may e replaced by a pseudo-peptide,
or a combination of such modifications,
provided that
when the PTH compound is free from D- or L-α-amino acid attached to the N-terminus or from C₂₋₆alcoxycarbonyl or optionally substituted C₁₋₈alkyl, C₂₋₈alkenyl, C₂₋₈alkynyl, aralkyl, C₂₋₈alkynyl, aralkenyl or C₃₋₆cycloalkyl-C₁₋₄alkyl, it is other than a PTH compound having a naturally occurring α-amino acid sequence;
or the PTH compound is other than PTH(1-34) wherein
i. the α-amino acid in position 1 is Gly, D-Ser, D-Ala or Tyr;
or
ii. the α-amino acid in position 2 is Ala, D-Val, Lys, Arg or Cit and the α-amino acid in position 34 is Tyr; or the α-amino acid in position 2 is D-Val and α-amino acid in position 34 is D-Tyr and optionally the α-amino acids in positions 8 and 18 are each Nle; or
iii. the α-amino acid in positions 3 and/or 6 and/or 9 are replaced by a natural or unnatural amino acid; or
iv. the α-amino acid in position 23 is replaced by Ala, Arg, Asn, Asp, Cys, Gln, Glu, Gly, His, Ile, Lys, Met, or Thr; or
v. the α-amino acid in position 25 and/or 26 and/or 27 is replaced by Ala, Asn, Asp, Cys, Gln, Glu, Gly, His, Ile, Leu, Ser, Thr, Trp, Tyr or Val; or
vi. the α-amino acids in positions 8 and 18 are each Nle or each Met (O) optionally the α-amino acid in position 34 is Tyr; or the α-amino acids in positions 8 and 18 are each Nle and the α-amino acid in position 34 is Tyr and either the α-amino acid in position 12 is L- or D-pro, L- or D-Ala, Aib or NMeGly or the α-amino acid in position 23 is Phe, Leu, Nle, Val, Tyr, α-Nal or β-Nal; or
vii. the α-amino acid in position 28 is Lys and the α-amino acid in position 30 is Leu; or
viii. the α-amino acid in position 1 is or the α-amino acid in position 8 and/or 18 is Leu, Ile, Val, Phe or Trp; and/or the α-amino acid in position 11 is Ser, Lys, Phe, β-Nal, Trp or Tyr, and/or the α-amino acid in position 12 is D-Leu, D-Ile, D-Nle, D-Ser, D-Ser(Butyl), D-Abu, D-Thr, D-Nva, Met, D-β-Nal, D-Trp, D-Lys, D-Tyr, D-Lys(Fmoc), D-Phe or D-Asn; and/or the α-amino acid in position 13 is Leu; and/or the α-amino acid in position 19 in position 21 is Arg, Lys, Asn or His; and/or the α-amino acid in position 23 is 2-(1,3-dithiolane-2-yl)Trp; and/or the α-amino acid in position 25 and/or in position 26 is His; and/or the α-amino acid in position 27 is Gln or Leu; or
ix. the α-amino acid in position 8 and/or 18 is Ala or Ser; or the α-amino acid in position 8 and/or 18 is Ala, Val, Leu, Ile, Ser or Trp and the α-amino acid in position 34 is Tyr; or
the PTH compound is an PTH(1-84) wherein
i. the α-amino acid in position 1 is Tyr, Val, Pro, Asp or Cys; or
ii. the α-amino acid in position 2 is Ala, Glu, Leu, Ser or Arg; or
iii. the α-amino acid in position 3 and/or 6 and/or 9 are replaced by a natural or unnatural amino acid; or
iv. the α-amino acid in position 2 Ala, Arg, Asn, Asp, Cys, Gln, Glu, Gly, His, Ile, Lys, Met, Pro, Ser or Thr; or
v. the α-amino acid in position or 26 and/or 27 is replaced by Ala, Asn, Asp, Cys, Gly, His, Ile, Leu, Met, Phe, Pro, Tyr or Val; or
vi. the α-amino acid in position 8 is Met, Met(O), Ala, Val, Leu, Ile, Ser, Trp, Asn, Gln, Asp, Glu, Lys, Arg, Tyr or Gly and the α-amino acid in position 18 is Leu; or the α-amino acid in position 8 and/or 18 is Ala, Val, Leu, Ile, Ser or Trp, and optionally the α-amino acid in position 34 is Tyr; or the α-amino acid in position 8 and in position 18 are each Met(O); or
vii. the α-amino acid in position 26 is Gln;
the compound other than Pro⁰PTH(1-84) or [Met⁰,Leu⁸,Leu¹⁸]PTH(1-84); or
the PTH compound is other than hPTH(1-36) wherein the α-amino acid in position 36 is Leu;
in free form or in salt form or complex form.

2. The PTH compound according to claim 1 comprising at least one amino acid unit replaced in one of the following positions of the PTH sequence: 1, 2, 3, 8-11, 13 to 19, 21, 22, 29 to 34.

3. The PTH compound according to claim 1 wherein the α-amino acid units in positions 1 and 2 at the amino terminus of the PTH sequence is replaced by a pseudo-dipeptide.

4. The PTH compound according to claim 1 of formula I
X—P₁ I
wherein
X is a residue of formula (a) 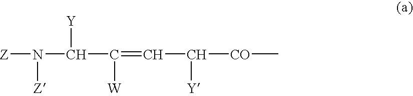
or (b) 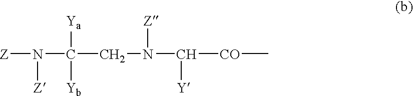
wherein each of Z and Z′, independently, is H; optionally substituted C₁₋₈alkyl, C₂₋₈alkenyl, aralkyl, aralkenyl or C₃₋₆cycloalkyl-C₁₋₄alkyl; or a protecting group, at most Z and Z′ being a protecting group,
Z″ is H, C₁₋₈alkyl or a protecting group
each of Y and Y′, independently, is an optionally protected side chain of a natural or unnatural α-amino acid,
one of Yₐ and Y_{b} is hydrogen and the other is an optionally protected side chain of a natural or unnatural α-amino acid or Yₐ and Y_{b} form together with the carbon atom to which they are attached a C₃₋₆cycloalkyl group,
and
W is halogen or C₁₋₄alkyl,
or W and Y′ together with the moiety 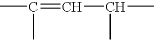
to which they are attached, form an optionally substituted aromatic cyclic or heterocyclic residue, and
P₁ is a PTH sequence in which amino acid units in positions 1 and 2 of the N-terminus are omitted.

5. The PTH compound according to claim 1, wherein the α-amino acid unit in position 1 and/or the α-amino acid unit in position 2 of the N-terminus of the PTH sequence is replaced by an optionally protected natural or unnatural amino acid residue, provided that
when the PTH compound is free L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;
or the PTH compound is other than PTH(1-34) wherein
i. the α-amino acid in position 1 is Aib, Gly, D-Ser, or Tyr; or
ii. the α-amino acid in position 2 is Ala, D-Val, Lys, or Arg and the α-amino acid in position 34 is Tyr; or the α-amino acid in position 2 is D-Val and the α-amino acid in position 34 is D-Tyr and optionally the α-amino acids in positions 8 and 18 are each Nle; or
iii. the α-amino a position 1 is Aib and at least acid unit has been replaced as follows: the α-amino acid in position 8 and/or 18 is Leu, Ile, Val, Ph or Trp, and/or the α-amino acid in position 11 is Ser, Lys, Phe, β-Nal, Trp or Tyr, and/or the α-amino acid in position 12 is D-Leu, D-Ile, D-Nle, D-Val, D-Ser, D-Ser(Butyl), D-Abu, D-Thr, D-Nva, D-Met, D-Nal, D-Trp, D-Lys, D-Tyr, D-Lys(Fmoc), D-Phe or D-Asn, and/or the α-amino acid in position 13 is Leu, and/or the α-amino acid in position 19 and/or in position 21 is Arg, Lys, Asn or His, and/or the α-amino acid in position 23 is 2-(1,3-dithiolane-2-yl)Trp, and/or the α-amino acid in position 25 and/or in position 26 is His, and/or the α-amino acid in position 27 is Gln or Leu,
or the PTH compound is other than PTH(1-84) wherein
i. the α-amino acid in position 1 is Tyr, Val, Pro, Asp or Cys; or
ii. the α-amino acid in position 2 is Ala, Glu, Leu, Ser or Arg.

6. The PTH compound according to claim 1 of formula II
X₁ₐ—Y₂ₐ—P₂ II
wherein
X₁ₐ is a residue of an optionally protected natural α-amino acid or a residue of formula (IIa) 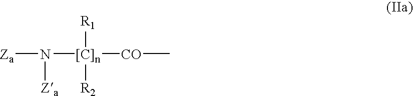
wherein
each of Zₐ and Z′ₐ, independently, is H, C₁₋₆alkyl or a protecting group, at most one of Zₐ and Z′ₐ being a protecting group, and either n is 1 and
i. each of R₁ and R₂, independently, is C₁₋₆alkyl, or
ii. one of R₁ and R₂ is methyl or ethyl and the other is an optionally protected residue attaching to the α-carbon atom of a natural α-amino acid other than Ala, Leu, Ile or Val, or
iii. one of R₁ and R₂ is H, methyl or ethyl and the other is an optionally protected residue attaching to the α-carbon atom of an unnatural α-amino acid, or
iv. R₁ and R₂ form together with the carbon atom to which they are attached a C₃₋₆cycloalkyl group, or
v. the residue 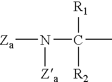
represents a heterocyclic residue optionally condensed to a benzene ring,
or n is 2, 3, 4 or 5 and each of R₁ and R₂ is H or CH₃,
Y₂ₐ is a residue of an optionally protected natural α-amino acid or a residue of formula (IIb) 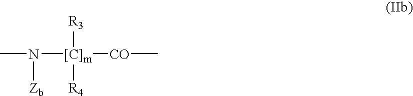
Z_{b} is H or C₁₋₆alkyl and either m is 1 and
i. each of R₃ and R₄, independently, is C₁₋₆alkyl, or
ii. one of R₃ and R₄ is methyl or ethyl and the other is an optional protected residue attaching to the α-carbon atom of a natural α-amino acid other than Ala, Leu, Ile Val, or
iii. one of R₃ and R₄ is H, methyl or ethyl and the other is an optionally protected residue attaching to the α-carbon atom of an unnatural α-amino acid,
or m is 2, 3, 4 or 5 R₃ and R₄ is H or CH₃, and
P₂ is a PTH sequence in which amino acid units in positions 1 and/or 2 of the N-terminus are omitted.

7. The PTH compound according to claim 1 wherein the α-amino acid unit in position 8 and/or the acid unit in position 18 is replaced by an optionally protected natural or unnatural amino acid residue, provided that
when the PTH compound is free from D- or L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;
or other than PTH(1-34) wherein
i. the α-amino acids in positions 8 and 18 are each Nle or each Met (0) and optionally the α-amino acid in position 34 is Tyr; or the α-amino acids in positions 8 and 18 are each Nle and the α-amino acid in position 34 is Tyr and either the α-amino acid in position 12 is L- or D-Pro, L- or D-Ala, Aib or NMeGly or the α-amino acid in position 23 is Phe, Leu, Nle, Val, Tyr, α-Nal or β-Nal; or the α-amino acids in positions 8 an 18 are each Nle and the α-amino acid in position 2 is D-Val and the α-amino acid in position 34 is D-Tyr; or
ii. the α-amino acid in position 8 and/or 18 is Leu, Ile, Val, Phe or Trp and optionally at least one further α-amino acid unit has been replaced as follows: the α-amino acid in position 1 is Aib and/or the α-amino acid in position 11 is Ser, Lys, Phe, β-Nal, Trp or Tyr, and/or the α-amino acid in position 12 is D-Leu, D-Ile, D-Nle, D-Val, D-Ser, D-Ser(Butyl), D-Abu, D-Thr, D-Nva, D-Met, D-β-Nal, D-Trp, D-Lys, D-Tyr, D-Lys(Fmoc), D-Phe or D-Asn, and/or the α-amino acid in position 13 is Leu, and/or the α-amino acid in position 19 and/or in position 21 is Arg, Lys, Asn or His, and/or the α-amino acid in position 23 is 2-(1,3-dithiolane-2-yl)Trp, and/or α-amino acid in position 25 and/or in position 26 is His, and/or the α-amino acid in position 27 is Gln or Leu; or
ii. the α-amino acid in position 8 and/or 18 is Ala or Ser; or the α-amino acid in position 8 and/or 18 is Leu, and the α-amino acid in position 34 is Tyr; or
the PTH compound is other than PTH(1-84) wherein the α-amino acid in position 8 is Met, Met(O), Ala, Val, Leu, Ile, Ser, Trp, Asn, Gln, Asp, Glu, Lys, Arg, Gly and the α-amino acid in position 18 is Leu; or the α-amino acid in position 8 and in position 18 are each Met(O); or the α-amino acid in position 8 is Leu and the α-amino acid in position 18 is Met(O); or
the PTH compound is other than [Leu¹⁸,Tyr³⁴]hPTH(1-84), [Leu⁸,Tyr³⁴]hPTH(1-84), [Ile⁸,Leu¹⁸,Tyr³⁴]hPTH(1-84) or [Leu⁸,Leu¹⁸,Tyr³⁴]hPTH(1-84).

8. A PTH compound having PTH-like activity wherein at least one of the α-amino acid unit in the positions 1 to 38 of a PTH sequence is replaced by the α-amino acid unit which is present at the corresponding position in PTHrP, the PTH compound optionally comprising at least one radical selected from a L- or D-α-amino acid, C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to the terminal amino group of the PTH compound, and/or at least one radical selected from C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to one or more side chain amino group of the PTH compound, provided that
when the PTH compound is free from D- or L-α-amino acid attached to the N-terminus or from C₂₋₆alkoxycarbonyl or optionally substituted C₁₋₈alkyl, C₂₋₈alkenyl, C₂₋₈alkynyl, aralkyl, aralkenyl or C₃₋₆cycloalkyl-C₁₋₄alkyl, it is other than a PTH compound having a naturally occurring α-amino acid sequence;
or the PTH compound is other than PTH(1-34) wherein
i. the α-amino acid in position 8 and/or 18 is Leu; or the α-amino acid in position 11 is Lys;
and/or the α-amino acid in position 19 and/or 21 is Arg; and/or the α-amino acid in position 25 and/or 26 is His; and/or the α-amino acid in position 27 is Leu; and optionally at least one further α-amino acid unit has been replaced as follows: the α-amino acid in position 1 Aib and/or the α-amino acid in position 11 is Ser, Phe, β-Nal, Trp or Tyr, and/or the α-amino acid in position 12 is D-Leu, D-Ile, D-Nle, D-Val, D-Ser, D-Ser(Butyl), D-Abu, D-Thr, D-Nva, D-Met, D-β-Nal, D-Trp, D-Lys, D-Tyr, D-Lys(Fmoc), D-Phe or D-Asn, and/or the α-amino acid in position 13 is Leu, and/or the α-amino acid in position 19 and/or in position 21 is Lys, His, and/or the α-amino acid in position 23 is 2-(1,3-dithiolane-2-yl)Trp, and/or the α-amino acid in position 27 is Gln; or
ii. the α-amino acid in position 25 in Gln and/or the Cα-amino acid in position 26 is Asn, the α-amino acid in position 27 being optionally Leu; or
the PTH compound is other than [Leu⁸,Leu¹⁸]PTH(1-84), in free form or in salt or complex form.

9. The PTH compound according to claim 8 wherein at least one of the α-amino acid units in the positions 8 to 11 of a PTH sequence is replaced by the α-amino acid unit which is present at the corresponding position in PTHrP.

10. The PTH compound according to claim 8 wherein at least one of the α-amino acid units in the positions 16 to 19 of a PTH replaced by the α-amino acid unit which is present at the corresponding position in PTHrP.

11. The PTH compound according to claim 8 wherein at least one of the α-amino acid units in the positions 33 and 34 of a PTH sequence is replaced by the α-amino acid unit which is present at the corresponding position in PTHrP.

12. The PTH compound according to claim 8 wherein one or more α-amino acid units in the remaining positions 1 to 38 is further replaced by a natural or unnatural amino acid unit.

13. The PTH compound according to claim 12 wherein the α-amino acid in position 10 is selected from Gly, Gln, Glu, His, Ser, Thr, Lys and Tyr, and/or the α-amino acid in position 13 is a D- or L-α-amino acid other than Arg, and/or the α-amino acid in position 16 is a D- or L-α-amino acid, and/or the α-amino acid in position 18 is Gln or Tyr, and/or the α-amino acid in position 19 is selected from Ala, Arg, Val, Tyr, Ser, Lys, Met, His, Gly, Pro, Asn and Ile, and/or the α-amino acid in position 33 is a D- or L-α-amino acid other than Thr, Phe or Ala.

14. The PTH compound according to claim 8 which is selected from [Leu⁸, Gln¹⁸, Thr³³, Ala³⁴]PTH, [Leu⁸, Ala¹⁶, Gln¹⁸, Thr³³, Ala³⁴]PTH, [Leu⁸, Asp¹⁰, Lys¹¹, Ala¹⁶, Gln¹⁶, Thr³³ Ala³⁴]PTH, [Leu⁸, Asp¹⁰, Lys¹¹, Ala¹⁶, Gln¹⁸]PTH, [Leu⁸, Ala¹⁶, Gln¹⁸, Ala¹⁹, Thr³³, Ala³⁴]PTH, [Leu⁸, Asp¹¹, Lys¹¹, Gln¹⁸]PTH, [Leu⁸, Asp¹⁰, Lys¹¹, Ala¹⁶, Gln¹⁸, Ala¹⁹]PTH, [Leu⁸, Ala¹⁶, Gln¹⁸, Ala¹⁹, Thr³³, Ala³⁴]PTH and [Leu⁸, Asp¹⁰, Lys¹¹, Gln¹⁸, Thr³³, Ala³⁴]PTH, the PTH compound optionally comprising at least one radical selected from a L- or D-α-amino acid, C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to the terminal amino group of the PTH compound, and/or at least one radical selected from C₂₋₆alcoxycarbonyl and optionally substituted C₁₋₈alkyl, C₂₋₈alkenyl, C₂₋₈alkynyl, aralkyl, aralkenyl or C₃₋₆cycloalkyl-C₁₋₄alkyl and attached to one or more side chain amino groups of the PTH compound, in free form or in salt or complex form.

15. The PTH compound according to claim 1 which is a human N-terminal PTH fragment comprising 1-34 to 1-38 amino acid units.

16. The PTH compound according to claim 1 wherein the C-terminus is —COOH, esterified —COOH, —CONH₂, mono- or disubstituted —CONH₂.

17. A PTH compound selected from
[Leu⁸,Gln¹⁸,Thr³³,Ala³⁴]-hPTH(1-34)OH
[Leu⁸,Ala¹⁶,Gln¹⁸,Ala¹⁹,Thr³³,Ala³⁴]hPTH(1-34)OH
[Leu⁸,Ala¹⁶,Gln¹⁸,Thr³³,Ala³⁴]hPTH(1-34)OH
[Leu⁸,Asp¹¹,Lys¹¹,Gln¹⁸]hPTH(1-36)OH
[Leu⁸,Asp¹¹,Lys¹¹,Gln¹⁸,Thr³³,Ala³⁴]hPTH(1-34)OH
[Leu⁸,Asp¹⁰,Lys¹¹,Ala¹⁶,Gln¹⁸,Ala¹⁹]hPTH(1-36)OH
[Leu⁸,Asp¹⁰,Lys¹¹,Ala¹⁶,Gln¹⁸,Thr³³,Ala³⁴]hPTH(1-34)OH
[Leu⁸,Asp¹⁰,Lys¹¹,Ala¹⁶,Gln¹⁸]hPTH(1-36) OH, and
[Leu⁸,Asp¹⁰,Lys¹¹,Ala¹⁹]hPTH(1-36)OH
in free form or in salt or complex form.

18. A process for the production of a desired medium-sized polypeptide in which a fusion protein comprising an N-terminal bacteriophage T4 gene 55 polypeptide having the desired polypeptide linked to the C-terminal thereof, is expressed in bacterial cells.

19. The process according to claim 18 in which the fusion protein comprises a chemically cleavable linker between the gene 55 and the desired polypeptides comprising the amino acid sequences Asp-Pro-Pro or Asn-Gly-Pro.

20. A nucleotide sequence which codes for a fusion protein comprising an N-terminal bacteriophage T4 gene 55 polypeptide and a desired medium-sized polypeptide linked to the C-terminal thereof.

21. A bacterial expression vector comprising a DNA sequence according to claim 20.

22. A bacterial host cell transformed with the DNA sequence according to claim 20.

23. A fusion protein comprising an N-terminal bacteriophage T4 gene 55 polypeptide and a desired medium-sized polypeptide linked to the C-terminal thereof.

24. The process according to claim 18 in which the desired medium-sized polypeptide comprises a PTH polypeptide.

25. The process according to claim 18 comprising:
culturing bacterial host cells transformed with a vector containing a DNA sequence coding for expression of a fusion protein comprising an N-terminal bacteriophage T4 polypeptide having the desired polypeptide linked to the C-terminal thereof by a chemically cleavable linker selected from the group consisting of peptides containing the amino acid sequence Asp-Pro-Pro or Asn-Gly-Pro and expressing the fusion protein in the form of inclusion bodies;
isolating the inclusion bodies;
solubilising the inclusion bodies under acid conditions;
cleaving the desired polypeptide from the fusion protein, and
removing unwanted N-terminal amino acid residues from the desired polypeptide product.

26. A pharmaceutical composition comprising a PTH compound according to claim 1, in free form or in pharmaceutically acceptable salt or complex form, together with a pharmaceutically acceptable diluent or carrier therefor.

27. The method for improving bone formation in a subject in need of such treatment, which comprises administering to said subject an effective amount of PTH compound according to claim 1, in free form or in pharmaceutically acceptable salt or complex form.

28. The method according to claim 27, further comprising administering to said subject an effective amount of a second drug substance which is a bone resorption inhibitor.

29. A compound of formula IIaa or IIbb
ZZ′N—CHY—CW═CH—CHY′—COOH (IIaa)
ZZ′N—CYₐY_{b}—CH₂—NZ″-CRY′—COOH (IIbb)
wherein each of Z and Z′, independently, is H; optionally substituted C₁₋₈alkyl, C₂₋₈alkenyl, aralkyl, aralkenyl or C₃₋₆cycloalkyl-C₁₋₄alkyl; or a protecting group, at most one of Z and Z′ being a protecting group,
Z″ is H, C₁₋₈alkyl or a protecting group
each of Y and Y′, independently, is an optionally protected side chain of a natural or unnatural α-amino acid,
one of Yₐ and Y_{b} is hydrogen and the other is an optionally protected side chain of a natural or unnatural α-amino acid or Yₐ and Y_{b} form together with the carbon atom to which they are attached a C₃₋₆cycloalkyl group,
and
W is halogen or C₁₋₄alkyl,
or W and Y′ together with the moiety 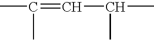
to which they are attached, form an optionally substituted aromatic cyclic or heterocyclic residue.


SEQUENCE LISTING (18 sequences)

SEQ ID NO: 1 - DNA, 27 bp; single-stranded, linear
  hypothetical: NO
  anti-sense: NO
gaggtgcatatgtcagaaactaagcct

SEQ ID NO: 2 - DNA, 40 bp; single-stranded, linear
  hypothetical: NO
  anti-sense: NO
ggtcggatccatcgttagcgttagcctcatataaaaaatc

SEQ ID NO: 3 - DNA, 28 bp; single-stranded, linear
  hypothetical: NO
  anti-sense: NO
ggtcggatcctacgttggacgaatgcat

SEQ ID NO: 4 - DNA, 42 bp; single-stranded, linear
  hypothetical: NO
  anti-sense: NO
gagtggatccatcgatccaccatccgtatcagaaatacaact

SEQ ID NO: 5 - DNA, 42 bp; single-stranded, linear
  hypothetical: NO
  anti-sense: NO
gagtggatccgttaacggtccatccgtatcagaaatacaact

SEQ ID NO: 6 - DNA, 34 bp; single-stranded, linear
  hypothetical: NO
  anti-sense: NO
tctactcgagttaacccagagctacaaaattatg

SEQ ID NO: 7 - DNA, 717 bp; single-stranded, linear
  CDS 101..562
  hypothetical: NO
  anti-sense: NO
agatctcgatcccgcgaaattaatacgactcactatagggagaccacaacggtttccctctagaaataattttgtttaactttaagaaggagatatacatatgtcagaaactaagcctaaatataattacgtaaacaataaagagcttttacaagctattattgattggaaaacagaattagcaaataataaagacccaaataaagtagttcgtcagaatgatactatcggattagccattatgcttattgcagaaggcttatctaaacgtttcaacttttcaggatacacccagtcttggaaacaagaaatgattgcagatggtatagaagcttctattaaggggcttcacaattttgatgaaacgaaatataaaaacccacatgcgtatataactcaagcttgttttaatgcattcgtccaacgtggatccatcgatccaccatccgtatcagaaatacaactaatgcataatctgggtaaacatctgaattcaatggaacgtgtagaatggctgcgtaaaaaactgcaggatgtacataattttgtagctctgggttaactcgagcagatccggctgctaacaaagcccgaaaggaagctgagttggctgctgccaccgctgagcaataactagcataaccccttggggcccttggggcctctaaacgggtcttgaggggttttttgctgaaaggaggaactatatccggataa

SEQ ID NO: 8 - DNA, 945 bp; single-stranded, linear
  CDS 101..790
  hypothetical: NO
  anti-sense: NO
agatctcgatcccgcgaaattaatacgactcactatagggagaccacaacggtttccctctagaaataattttgtttaactttaagaaggagatatacatatgtcagaaactaagcctaaatataattacgtaaacaataaagagcttttacaagctattattgattggaaaacagaattagcaaataataaagacccaaataaagtagttcgtcagaatgatactatcggattagccattatgcttattgcagaaggcttatctaaacgtttcaacttttcaggatacacccagtcttggaaacaagaaatgattgcagatggtatagaagcttctattaaggggcttcacaattttgatgaaacgaaatataaaaacccacatgcgtatataactcaagcttgttttaatgcattcgtccaacgtattaaaaaagaacgtaaggaagttgcaaagaaatatagttacttcgttcacaatgtctatgacagccgtgacgacgatatggttgcgttagtagatgaaacttttattcaagacatctatgataaaatgacgcattacgaagaatcaacctatagaacaccgggggctgaaaagaaaagtgttgtagatgattctcctagtttggattttttatatgaggctaacgatggatccgttaacggtccatccgtatcagaaatacaactaatgcataatctgggtaaacatctgaattcaatggaacgtgtagaatggctgcgtaaaaaactgcaggatgtacataattttgtagctctgggttaactcgagcagatccggctgctaacaaagcccgaaaggaagctgagttggctgctgccaccgctgagcaataactagcataaccccttggggcccttggggcctctaaacgggtcttgaggggttttttgctgaaaggaggaactatatccggataa

SEQ ID NO: 9 - protein, 4 aa
  Region 1..4: /note= “Sequence is in position 16 to 19 of human parathyroid hormone (hPTH), hPTH (1-36), and hPTH (1-34).”
  hypothetical: NO
  fragment type: internal
QDLR

SEQ ID NO: 10 - protein, 6 aa
  Region 1..6: /note= “Sequence is in position 14 to 19 of hPTH (1-36) and hPTH (1-34)NH-2.”
  hypothetical: NO
  fragment type: internal
SIQDLR

SEQ ID NO: 11 - protein, 4 aa
  Region 1..4: /note= “Sequence is in position 16 to 19 of hPTH (1-36).”
  hypothetical: NO
  fragment type: internal
ADLA

SEQ ID NO: 12 - protein, 4 aa
  Region 1..4: /note= “Sequence is in position 16 to 19 of hPTH (1-36).”
  hypothetical: NO
  fragment type: internal
ADQA

SEQ ID NO: 13 - protein, 4 aa
  Region 1..4: /note= “Sequence is in position 16 to 19 of hPTH (1-36).”
  hypothetical: NO
  fragment type: internal
AAQA

SEQ ID NO: 14 - protein, 4 aa
  Region 1..4: /note= “Sequence is in position 16 to 19 of hPTH (1-36).”
  hypothetical: NO
  fragment type: internal
AAAA

SEQ ID NO: 15 - protein, 7 aa
  Region 1..7: /note= “Sequence is in position 25 to 31 of hPTH (1-34)NH-2.”
  hypothetical: NO
  fragment type: internal
HHLIAEI

SEQ ID NO: 16 - protein, 84 aa
  hypothetical: NO
SVSEIQLMHNLGKHLNSMERVEWLRKKLQDVHNFVALGAPLAPRDAGSQRPRKKEDNVLVESHEKSLGEADLADVNVLTKAKSQ

SEQ ID NO: 17 - protein, 153 aa
MSETKPKYNYVNNKELLQAIIDWKTELANNKDPNKVVRQNDTIGLAIMLIAEGLSKRFNFSGYTQSWKQEMIADGIEASIKGLHNFDETKYKNPHAYITQACFNAFVQRGSIDPPSVSEIQLMHNLGKHLNSMERVEWLRKKLQDVHNFVALG

SEQ ID NO: 18 - protein, 229 aa
MSETKPKYNYVNNKELLQAIIDWKTELANNKDPNKVVRQNDTIGLAIMLIAEGLSKRFNFSGYTQSWKQEMIADGIEASIKGLHNFDETKYKNPHAYITQACFNAFVQRIKKERKEVAKKYSYFVHNVYDSRDDDMVALVDETFIQDIYDKMTHYEESTYRTPGAEKKSVVDDSPSLDFLYEANDGSVNGPSVSEIQLMHNLGKHLNSMERVEWLRKKLQDVHNFVALG